(19) United States
(12) Patent Application Publication
(10) Pub. No.: US 2002/0076401 A1
(43) Pub. Date: Jun. 20, 2002

(54) Heat shock protein inducing factor and method for its in-situ generation from an inactive form

(76) Inventor: Vladimir Volloch, Brookline, MA (US)

Correspondence Address: David Prashker, Esq., DAVID PRASHKER, P.C., P.O. Box 5387, Magnolia, MA, 01930, US

(21) Appl. No.: 09/727,938

(22) Filed: Nov. 30, 2000

Related U.S. Application Data
(60) Provisional application No. 60/234,406, filed on Sep. 21, 2000.

(51) Int. Cl. A61K 38/43; A61K 31/185

(52) U.S. Cl. 424/94.1; 514/553

(57) ABSTRACT

The present invention provides an active heat shock protein inducing factor which is capable of inducing expression of heat shock proteins such as Hsp70 and Hsp27 on-demand in living mammalian cells. The induction and intracellular expression of such heat shock proteins provides major protection for the cells against injurious stresses and can be used therapeutically for treatment of conditions and disorders caused by cell death via apoptosis or necrosis.

The heat shock protein-inducing factor is a naturally occurring molecule to be found in the blood serum of living mammals in two different states and activity forms: an inactive form which circulates freely and systemically as a component of the blood serum; and an active molecule which is generated by conversion from the inactive form via modification in-situ. Such a conversion occurs physiologically as a result of stresses or may be effected in-situ ondemand. The invention includes the means and method for converting the inactive form into the active heat shock protein inducing factor state on-demand under both in-vivo and in-vitro circumstances.

### PROVISIONAL PATENT APPLICATION

This invention was first filed as a Provisional Patent Application on Sep. 21, 2000 as U.S. Serial No. 60/234,406.

### FIELD OF THE INVENTION

The present invention is concerned generally with heat shock proteins (HSPs) and the benefits and advantages provided by the intracellular expression of heat shock proteins such as Hsp70 (also known as Hsp72) and Hsp27; and is directed in particular to the identification, characterization, and activation of a heat shock protein inducing factor (HIF) capable of inducing expression of heat shock proteins on-demand within living mammalian cells and organisms.

### BACKGROUND OF THE INVENTION

In all living organisms, there is a common survival mechanism called heat shock or the stress response (also known as heat shock response because of its first detection as a response to heat shock), which enhances cellular resistance to and serves as the major mechanism of cellular defense against damaging stresses [Welch, W. J., Physiol. Rev. 77: 1063-1081 (1992); Parsell, D. A. and S. Lindquist, “Heat Shock Proteins And Stress Tolerance”, in The Biology Of Heat Shock Proteins And Molecular Chaperones (Morimoto, R. I., Tissieres, A., & Georgopulos, eds.), Cold Spring Harbor Laboratory Press, 1994; Cotto, J. J. and R. I. Morimoto, Biochem. Soc. Symp. 64: 105-108 (1999); Jaattela, M., Ann. Med. 31: 261-271 (1999); and Morimoto, R. I. and M. G. Santoro, Nat. Biotechnol. 16: 833-838 (1998)]. The induction of this stress response protects, among other things, against the initial damaging insult; augments functional cell recovery; and confers transient resistance to subsequent cellular stress of different types. This stress response and subsequent cellular resistance also correlate with the de novo synthesis of a group of proteins known collectively as heat shock proteins (HSPs).

In-vitro and in-vivo evidence both demonstrate that induction of a stress response by the synthesis of HSPs protects, among other things, against ischemic injury, oxidative stress, and other stresses resulting in programmed cell death (apoptosis) and even in necrotic cell death. Interestingly, when expressed in cells or organs, expression of even a single species of HSP (such as the major stress-inducible species Hsp70) is sufficient to reduce by more than 70% the damage to cells by oxidative ischemic stress [Rjdev. et al., Ann. Neurol. 47: 782-791 (2000); Plumier et. al., J. Clin. Invest. 95: 1854-1860 (1995); Radford et. al., Proc. Natl. Acad. Sci. USA 93 2339-2342 (1996); Hutter et. al., Circulation 94:1408-1411 (1996); Plumier et. al., Cell Stress Chaperones 2: 162-167 (1997). To achieve such effective protection, HSPs such as Hsp70 interact at multiple levels of the cell death cascade. Hsp70 reduces or inhibits necrosis as well as caspase-dependent and Bindependent delayed (programmed) cell death. The stress response typically not only induces Hsp70 expression but also the synthesis of many other HSPs, such as Hsp27, with additional protective properties against necrotic and apoptotic cell death. However, Hsp70 is considered to be essential and, in many cases, is itself sufficient for protection against stressful insults Therefore, the means to induce the stress response in general, and the expression of Hsp70 in particular, has great therapeutic potentials as a preventive treatment and/or as a strategy for treatment of a range of diseases—including, among others, stroke, neurodegenerative diseases, myocardial inefficiency, tissue damage and trauma, and other conditions caused by cell death via either apoptitc or necrotic pathways.

Induction of Cell Resistance:

Cells exposed to a brief heat stress acquire resistance to a subsequent lethal heat treatment as well as to a variety of other stressful stimuli such as ultraviolet radiation, treatment with chemotherapeutic drugs or amino acid analogs, oxidative stress or simulated ischemia. Conversely, cellular resistance to lethal heat treatment can also be conferred by many conditioning stresses. From these initial observations originate the general concepts of stress response and cell tolerance to stress: mild stressful stimuli induce a survival mechanism, the stress response, which confers cellular resistance to a large variety of lethal treatments [Welch, W. J., Physiol. Rev. 77: 1063-1081 (1992); Cotto, J. J. and R. I. Morimoto, Biochem. Soc. Symp. 64: 105-108 (1999); Jaattela, M., Ann. Med. 31: 261-271 (1999)]. The acquisition of the resistance induced by heat shock or other stressful treatments correlates with the synthesis of new proteins, accordingly called heat shock proteins, the major of which is Hsp70.

There are several multigene HSP families which are defined by the approximate molecular weight of their protein products [for review see Lindquist, S. and S. Craig, Ann. Rev. Genet. 22: 631-677 (1988)]. All HSPs share a common function that is to police cellular protein quality. Most HSPs are chaperone proteins used during the folding process or during membrane translocation, and therefore act to prevent protein misfolding and aggregation; as chaperones, they also assist in degradation of proteins damaged beyond repair. For example, Hsp70, Hsp40 and Hsp60 family members participate in nascent chain folding [Hartl, F., Nature 381: 571-579 (1996)]. In addition, Hsp70 members also contribute to the membrane translocation process; and small HSPs, such as Hsp27, trap and maintain denatured proteins in a folding-competent state. Hsp90 can participate in folding but mostly interacts with many regulatory proteins [Pratt, W., Annu. Rev. Pharmacol. Toxicol. 37: 297-326 (1997)].

While stress induces the synthesis of all HSPs by definition, the degree of expression for individual HSP species varies enormously, Hsp70 being by far the major and most inducible HSP. In mammalian cells, micro-injection of anti-Hsp70 antibodies reduced cell survival to heat [Riabowol et al., Science 242: 433-436 (1988)]. The same effect was observed when antisense Hsp70 RNA was expressed in cells [Volloch et. al., Cell Stress & Chaperones 3: 265-271 (1998); Volloch et. al., FEBS Lett. 461: 73-76 (1999); Volloch, V. and S. Rits, Exp. Cell Res. 253: 483-492 (1999); Volloch et. al., Cell Stress & Chaperones 5: 139-147 (1999)]. Convincingly, transformation of mammalian cells to constitutively express human Hsp70 gene increased cell tolerance to heat shock [Angelidis et al, Eur. J. Biochem. 199: 35-39 (1991); Li et al., Proc. Natl. Acad. Sci. USA 88: 11681-1685 (1991)]. Similarly, transfection of cells with constitutive Hsp27 genes conferred to cells an increased resistance to heat [Fortin et al., J. Radiat. Oncol. Biol. Phys. 46: 1259-1266 (2000)]. Therefore, it becomes apparent that individual HSPs have protective functions when expressed at high intracellular levels. Additional research studies demonstrate that the induction of a battery of different HSP types provide even more resistance to stress than the synthesis of a single HSP species. Indeed, there is evidence of some synergy between the various HSPs. For example, Hsp70 and Hsp40 interact; as do Hsp70 and Hsp90; as well as Hsp70 and Hsp27.

Mechanisms of HSP-Mediated Cell Protection:

Because of their chaperoning function, HSPs were proposed as a means to protect cells by preventing aggregation of proteins denatured by the stressful conditions and by assisting in the protein re-folding process. In-vitro cell-free analyses demonstrate that both Hsp70 and Hsp27 decrease protein aggregate formation and increase the re-folding rate and the functional recovery of heat-denatured enzymes. Interestingly, additional molecular targets in HSP-mediated protection have recently been demonstrated. For example, transfection experiments demonstrated that Hsp70 prevents apoptotic cell death. Thus, Hsp70 may act at several levels of the apoptotic pathway depending on the cell type and apoptotic stimulus. This approach is supported by the following: Firstly, Hsp70 can inhibit detrimental long-lasting activation of JNK induced by various stressors {Volloch et. al., FEBS Lett. 461: 73-76 (1999)]. Secondly, Hsp70 associates with BAG-l, the Bcl2-binding protein which enhances resistance to apoptosis. Thirdly, Hsp70 can block activation of caspase-3, one of the executors of cell death [Mosser et. al., Mol. Cell Biol. 17: 5317-5327 (1997)]. Fourthly, Hsp70 can also protect against cell death induced by caspase-3 enforced expression, suggesting that Hsp70 can prevent apoptosis at a late stage in the death signaling pathway [Jaattela et. al., EMBO 17: 6124-6134 (1998)]. Finally, two groups independently reported that Hsp70 inhibits the formation of the apoptosome by preventing interaction of procaspase-9 with APAF-1 [Beere et al., Nat. Cell Biol. 2: 469-475 (2000); Saleh et al., Natl. Cell Biol. 2: 476-483 (2000)]. All this evidence demonstrate that Hsp70 represents a major line of defense against cell injury: not only does Hsp70 contribute to cellular recovery by chaperoning damaged proteins, Hsp70 also prevents the completion of programmed cell death. Similarly, Hsp27 also protects from apoptotic cell death by binding to cytochrome c and blocking cytochrome c inhibition of the apoptosome [Bruey et al., Natl. Cell Biol. 2: 645-652 (2000); Garrido et. al., FASEB 13: 2061-2070 (1999)].

HSPs in Ischemia (As an Illustrative Example):

In-vivo, hyperthermia or other pathological conditions also trigger the stress response and HSP synthesis. For example, in focal cerebral ischemia, Hsp70 mRNA accumulates in the area at risk or can delineate a molecular penumbra where protein denaturation occurs [Plumier et. al., Cell Stress Chaperones 2: 162-167 (1997); Rajev et al., Ann. Neurol. 47: 782-791 (2000)]. Unfortunately, while Hsp70 mRNA rapidly accumulates in the ischemic area, Hsp70 protein is only detected hours after the beginning of the ischemic insult, much too late to prevent necrosis or cell commitment to delayed cell death. The role of Hsp70 in the stress response-mediated protection against myocardial ischemia was examined in transgenic mice carrying constitutively expresssed Hsp70 gene. Hsp70 expressing transgenic mice showed greatly reduced myocardial injury and increased functional recovery after ischemia [Plumier et al., J. Clin. Invest. 95: 1854-1860 (1995); Marber et al., J. Clin. Invest. 95: 1446-1456 (1995)].

There are several lines of evidence suggesting that a similar kind of Hsp70 protection could also be observed in the brain. Induction of HSPs in neuronal cultures reduced neuronal cell death following glutamate treatment [Rordorf et al., Neuron 7: 1043-1051 (1991)] or following oxygen-glucose deprivation to both neuronal and myocardial cells. Induction of Hsp70 in neurons using viral vector also confers protection against stimulated ischemia [Wagstaff et al., J. Biol. Chem. 274: 5061-5069 (1999); Rordorf et. al., Neuron 7: 1043-1051 (1991)].

In living animals, hyperthermia decreases the extent of damage after cerebral ischemia. Similarly, Hsp70 expression by mild ischemic treatment confers, also through induction of HSPs, tolerance to subsequent severe ischemia Additional evidence comes from the observation that striatal neurons and astrocytes transfected with Hsp70 gene using viral vectors have increased resistance to transient cerebral ischemia [Yenari et al., Mol. Med. Today 5: 525-531 (1999)]. Furthermore, the analysis of Hsp70 transgenic mice revealed that hippocampal neurons of Hsp70 transgenic mice were more resistant to ischemia-induced cell death [ Plumier et. al., Cell Stress Chaperones 2: 162-167 (1997)]. Finally, and convincingly, the infarct volumes measured 24 hours after transient or permanent occlusion of the middle cerebral artery were extraordinarily small in transgenic mice constitutively expressing Hsp70 (70-80% of reduction) [Rajdev et al., Ann. Neurol. 47: 782-791 (2000)]. All of this evidence clearly establishes that Hsp70-mediated protection against ischemic injury occurs not only in-vitro in various cell types, but also in-vivo both in the myocardium and in the brain.

Induction of the Stress Response:

The stress response is generally measured by the induction of the major HSP specie, Hsp70. Synthesis of Hsp70 is accomplished by transcriptional activation and preferential translation [Baeuerle et. al., Cell 87: 13-20 (1996); Morimoto, R. I., Science 1409-1410 (1993); Lis, J. and C. Wu, Cell 74:1-4 (1993); Wu, C., Rev. Cell Dev. Biol. 11: 441-469 (1995); Morimoto et. al., J. Biol. Chem. 267: 21987-21990 (1992); Abravaya et. al., Genes Dev. 6: 1153-1164 (1992); Shi et. al., Genes Dev. 12: 654-666 (1998); Cotto et. al., J. Biol. Chem. 271: 3355-3358 (1996); Sarge et. al., Mol. Cell Biol. 13: 1392-1407 (1993); Kline et. al., Mol. Cell Biol. 17: 2107-2115 (1997)]. One transcription factor, heat shock factor-1 (hsf-1) appears to be responsible for the induction of HSPs following hyperthermia and other stressful stimuli. Indeed, alterations of hsf-1 transcriptional activity such as observed in aged cells and senescent organisms reduce stress-induced expression of HSPs [Fawcett et. al., J. Biol. Chem. 269: 32272-32278 (1994)]. Moreover, targeted mutation of hsf-1 prevents hyperthermia-induced synethesis of Hsp70 and other HSPs. There are other factors such as hsf-2 and hsf-3 capable of regulating the expression of HSPs, but their mechanisms of action have not been fully elucidated. Activation of hsf-1 involves a feedback regulatory loop controlled by inhibitory interactions between hsf-1, Hsp70 and other HSPs.

There are several known means to induce HSP expression in-vivo. These involve either stresses or toxic or deleterious compounds. The most direct one is a mild heating of organs or tissues; but controlling the in-vivo temperature within a narrow therapeutic range can be difficult. In addition, the HSP induction threshold can vary between cell types. Alternatively, other approaches, such as systemic administration of kainic acid, can target Hsp70 specifically in subpopulations of neurons but major side effects are associated with the use of such compounds [Krueger et al., Brain Res. Mol. Brain Res. 71: 265-278 (1999)].

Recently, several compounds with Hsp70 expression enhancing properties have been described [for review, see Morimoto, R. I. and M. G. Santoro, Nat. Biotechnol. 16: 833-838 (1998); Smith et al., Pharmacol. Rev. 50: 493-514 (1998)]. Salivylates and non-steroidal anti-inflammatory drugs, initially believed to induce HSP synthesis, in fact only decreased the threshold requirements of the stress response induction. Similarly, hydroxylamine derivative (bimoclomol), although first reported as an Hsp70 inducer, was subsequently found to potentiate rather than activate the stress response. Other compounds including benzoquinoid ansamycins (such as herbimycin-A, geldanamycin and derivatives of geldanamycin) have been shown to activate Hsp70 synthesis through hsf-1 activation. These drugs (as well as an unrelated compound, radicicol) bind to Hsp90; and, by doing so, remove Hsp90-mediated inhibition of hsf-1. As a consequence, these drugs also block Hsp90 functions and interfere, with sometimes lethal consequences, with constitutive functions of Hsp90Xsuch as regulation of the steroid hormone receptor, several transcription factors, various tyrosine and serine/threonine kinases and nitric oxide synthase activity [for review see, Pratt, W. B. and D. O. Toft, Endocr. Rev. 18: 306-360 (1997); Pratt et. al., EXS 77: 79-95 (1996)]. Cyclopentone prostaglandins were also shown to induce Hsp70 synthesis for periods of 12-24 hours [de Marco et. al., Eur. J. Biochem. 256: 334-341 (1998)]. However, extended treatment with prostaglandins could have adverse side effects since prostaglandins contribute to various homeostatic functions, including maintenance of the gastric mucosa and electrolyte balance. Finally, both serine protease inhibitors and MG132, a proteasome inhibitor, induce HSP expression but cannot be used in-vivo because of their severe toxicity {Volloch et. al., Cell Stress & Chaperones 3:265-271 (1998)]. Therefore, all the HSP inducing strategies or inducers examined so far had either detrimental side effects or lacked potency in-vivo.

The capability to induce the stress response on-demand has enormous potential value as a therapeutic approach. The at-will induction of the stress response is medically beneficial because it triggers the synthesis of Hsp70 and many other HSPs with additional protective properties. Moreover, based on the considerable evidence demonstrating HSP-mediated protection against necrotic and apoptotic cell death, strategies to induce HSPs are of great therapeutic importance. Unfortunately, when tested in-vivo, all the conventionally known HSP-inducing candidates potentiate rather than activate the stress response or have major adverse side effects. Accordingly, while the goal of inducing the stress response on-demand is recognized as a most desirable therapeutic goal, no meaningfully effective method or mechanism has yet been demonstrated as meeting and satisfying this objective.

### SUMMARY OF THE INVENTION

The present invention provides a method for in-situ generation of an active heat shock protein inducing factor capable of inducing expression of at least one heat shock protein species, said method comprising the steps of:

obtaining access in-situ to a proteinaceous inactive form of heat shock protein-inducing factor, said proteinaceous inactive form being a naturally occurring composition in the blood serum of living mammals;

introducing an agent in sufficient quantity to said proteinaceous inactive form in-situ to initiate an activating reaction;

permitting said activating reaction to proceed in-situ such that active heat shock protein-inducing factor is generated as a reaction product, said active heat shock protein-inducing factor being able to induce cellular expression of at least one species of heat shock protein as a protection against injurious stress.

By definition, the term “in-situ” includes and comprises in-vivo, ex-vivo, and/or in-vitro circumstances and applications, as well as both human and veterinary uses and treatments.

### DESCRIPTION OF THE FIGURES

The present invention may be more easily understood and better appreciated when taken in conjunction with the accompanying drawing, in which:

FIGS. 1A and 1B are chromatographic immunoblottings cell fractions showing that Hsp-inducing factor present in ascites fluid induces Hsp72 expression in fibroblasts and myogenic cells of mammalian origin;

FIGS. 2A and 2B are chromatographic immunoblottings revealing that Hsp-inducing factor present in human and murine ascites fluids have similar effects on Hsp72 expression;

FIG. 3 is a chromatographic immunoblotting demonstrating that Hsp-inducing factor present in ascites fluid induces Hsp72 expression in murine lymphoma cells through a pathway different from the heat shock response pathway;

FIGS. 4A and 4B are chromatographic immunoblottings showing that the Hsp-inducing factor present in ascites fluid induces Hsp72 expression in aged cells that largely lost their ability to express Hsp72 in response to heat shock;

FIGS. 5A and 5B are chromatographic immunoblottings revealing that Hsp-inducing factor does not induce but does potentiate the expression of Hsp40 in either aged or young cells;

FIGS. 6A, 6B, and 6C are a graph and immunoblottings showing that Hsp-inducing factor acts to protect aged cells from injurious effects of heat shock stress;

FIG. 7 is a chromatographic immunoblotting demonstrating that Hsp-inducing factor in ascites fluid suppresses JNK activation in aged cells following severe heat shock stress;

FIG. 8 is a graph showing that HSP-inducing factor in ascites fluid protects aged mammalian cells from diverse stresses;

FIG. 9 is a graph showing that pretreatment with HIF protects primary neuronal cells from stresses;

FIG. 10 is a graph showing that HIF mediates induction of Hsp27;

FIG. 11 is a graph showing the restoration of stress response in aged cells by sub-Hsp70 inducing concentrations of HIF;

FIG. 12 is a graph illustrating in vivo activation in mouse serum by heat stree as a function of time;

FIG. 13 is a graph illustrating in vivo activation of HIF in rat serum by heat stress as a function of time;

FIG. 14 is a graph showing in vivo activation of HIF in mouse serum by surgical stress;

FIG. 15 is a graph illustrating in vitro activation of HIF in mouse serum by oxidizing treatment;

FIG. 16 is a graph illustrating in vivo activation of HIF in mouse serum by periodate injection;

FIG. 17 is a grap illustrating in vitro induction of Hsp70 expression in different mouse tissues by prolonged administration of periodate;

FIG. 18 is a graph showing that oxidizing treatment of serum from non-stressed mice or from variously stressed animals activates HIF to the same levels;

FIG. 19 is a graph illustrating that HIF from different sources induce Hsp70 production in cells incapable of Hsp70 expression in response to stresses; and

FIG. 20 is a graph illustrating that in vitro heat shock, but not HIF, induce expression of luciferase controlled by a truncated Hsp70 promoter.

### DETAILED DESCRIPTION OF THE INVENTION

The present invention identifies the existence and functional attributes of a heat shock protein inducing factor (HIF) which is capable of inducing expression of heat shock proteins (HSPs), among them Hsp70 and Hsp27 on-demand in living mammalian cells. The intracellular expression of such heat shock proteins provides major protection for the cells against injurious and detrimental stresses leading to cell death, such as for example, hypothermia, ischemia, oxidative stress, and hypoxia.

The present invention shows that heat shock protein inducing factor (HIF) is a naturally occurring, systemic molecule circulating in the blood serum of living mammals. HIF exists in two different forms: an inactive precursor (whose inactivity may be due either to chemical modification such as glycosilation or to association with an inhibitor); or an active molecule. By definition, a “precursor” is either an inactive molecule containing HIF as its integral part, or is a complex of HIF and an inhibitor which requires glycosilation or other modification for its inhibitory activity. The conversion from inactive to active state (which typically occurs physiologically as the result of a stress) can be achieved by deglycosilation (modification or removal of glycolic groups or any other groups attached to the protein via glycolic groups) of the HIF precursor, thus activating it; or of the HIF inhibitor, thus suppressing (deactivating) it. The means and method for converting the active precursor form into the active factor constitute a major aspect of the present invention. Accordingly both the heat shock protein inducing factor (HIF) and the method of converting the inactive precursor into the active form are described in detail hereinafter.

### I. The Heat Shock Protein Inducing Factor (HIF)

The experiments and empirical data presented hereinafter (part of which has been published as Volloch, V. and S. Rits, Exp. Cell Res. 253: 483-492 (1999)] demonstrate the occurrence and evidence the existence of a natural nontoxic extracellular factor, Hsp-inducing factor (HIF), capable of activating HSP species expression in different types of cells via a pathway distinct from the heat shock response pathway. Remarkably, HIF induces synthesis of Hsp70 in aged human cells that have lost the ability to express Hsp70 in response to heat shock and other stresses. Moreover, the presence of HIF in culture media protects aged cells from otherwise lethal stresses; and activation of HSP expression, such as Hsp70 expression, is essential for this protection.

The physiological role of HIF is therefore seen as a systemic regulator of heat shock protein (HSP) expression in response to physiological stresses. The HIF evaluated experimentally initially was a component of ascites fluid produced in response to peritoneal growth of myeloma (hybridoma) cells in mice or obtained from human patients. In addition, the HIF was evaluated experimentally as a component of blood serum activated following stresses, such as heat shock or surgical stress. Finally, the HIF evaluated experimentally was demonstrably converted from inactive to active form both in vitro and in vivo; and the evidence obtained showed that HIF from all sources mentioned above is one and the same.

HSP-Inducing Factor (HIF)

A. The identifying properties and functional attributes of HIF as a unique biochemical entity include the following:

(i) HIF is a proteinaceous factor and activator for inducing HSPs, including Hsp70 and Hsp27 expression in cells under both in-vivo and in-vitro conditions.

(ii) Cell incubation with HIF induces levels of Hsp70 and Hsp27 expression which are equal to the quantity of Hsp70 produced by heat shock treatment directly, and enhances the expression of Hsp40 in response to stress.

(iii) Induction of HSPs, including Hsp70 expression by HIF, occurs via a pathway distinct from the heat shock response pathway.

(iv) HIF induces HSPs, including Hsp70 expression, not only in young cells, but also in aged cells and other cell types incapable of synthesis of Hsp70 in response to stresses.

(v) The introduction and reactive presence of HIF in cultures of aged cells protects aged cells from apoptosis caused by otherwise lethal stresses.

(vi) Intentional introduction and in-situ treatment of cells with HIF is not itself stressful or harmful to the cells themselves. In-situ treatment comprises and individually includes cells to be found in-vivo, ex-vivo, and in-vitro.

(vii) HIF is a proteinaceous composition of matter which is activated in-situ from a precursor form; and appears in blood serum in active form quickly, typically within 5 minutes time after heat shock treatment (or 15-20 minutes after beginning heat treatment).

(viii) Once activated from its inactive form, HIF persists at maximal levels in serum for, typically, 30 minutes following the cessation of heat stress; then declines in concentration within about one hour=s time after activation; and declines to a negligible background level about two hours=time after the initial stressful event. Thus, heat shock protein inducing activity in-vivo (particularly for Hsp70 expression) appears quickly in circulation blood after a stress event or experience; is unstable in the activated form; and is removed from the blood relatively soon (about 2 hours) after the cessation of stress.

(ix) The naturally occurring inactive form of HIF is constitutively present in mammalian serum and is rapidly converted into the activated form on-demand under in-vivo conditions by heat shock or other kind of stress event.

(x) There is no de novo synthesis of an active heat shock protein-inducing factor as such in response to stress; rather the inactive form circulating in-vivo within the blood must be converted into the active form by an in-situ generating reaction process.

B. The chemical characteristics and properties of heat shock protein-inducing factor (HIF) as a composition of matter include the following:

(1) The precursor form comprises a glycoprotein (and possibly other groups attached to the protein via glycolic groups). Activation of the precursor occurs via oxidation and deglycosilation.

(2) Both the precursor and activated forms are proteinaceous entities with the active form having a molecular weight of about 57000 Da.

### II. The On-Demand Conversion of the Inactive Form to the Activated Form of Heat Shock Protein-Inducing Factor (HIF)

The novel stress-response inducing factor designated HIF (HSP-inducing factor) is a natural, physiological, extracellular (and to-date the only) known non-toxic compound capable of a robust induction of the major HSP cytoprotective proteins, Hsp70 among other HSPs. It should be recognized and appreciated that HIF activates Hsp70 expression in all cell lines and tissues tested to-date, indicating that it functions physiologically as a systemic regulator of Hsp70 expression. Remarkably also, it is capable of overcoming age-related attenuation of Hsp70 expression; and of inducing Hsp70; and restoring stress resistance even in aged human cells that have largely lost the ability to express this protein in response to stresses.

The naturally existing precursor for the active HIF capable of inducing the expression of Hsp70, Hsp27, and enhancing the expression of Hsp40 in response to stresses in all cell types tested (including cells normally incapable of a substantial expression of Hsp70 in response to stresses such as aged human cells or CH1 cells) is constitutively present in serum and in other physiological fluids, such as ascites fluid; and is present in inactive form. It is reiterated here that, by definition, the term “precursor” means either an inactive molecule containing HIF as its integral part; or a complex of HIF and its inhibitor. This form of precursor is rapidly activated in-vivo by a modifying agent(s) which is secreted into serum, or is present in serum constitutively (also in inactive form) and activated in-situ by the release of its cofactors under certain physiological circumstances such as stresses. Alternatively, inactive precursor is activated in-vivo by a modifying agent(s) which modify and thus inactivate an inhibitor associated with the HIF.

The inactive precursor can be converted in-vitro into the active inducing factor by treatment with sodium periodate (which is added to serum, ascites, or other physiological fluid) under the conditions of deglycosilation (removal from the protein of sugar groups) or by other deglycosilation treatments. In addition, the in-vivo administration of sodium periodate to live animals either via single injection or through prolonged delivery using an implanted pump converts the precursor in serum into the active factor; and such a conversion results in the induction of the expression of Hsp70 and Hsp27 in animal tissues. Finally, the obtained evidence shows that the Hsp72-inducing activity observed in ascites fluid, in serum following a stress, and generated by a periodate treatment either in vitro or in vivo are the same activity.

Accordingly, it will be recognized and appreciated that HSP-inducing factor is constitutively present within serum in inactive precursor form; and can be activated on-demand by modification or removal of glycosilic groups, or by the removal of other groups (for example lipids) attached to protein via glycosilic groups either on glycosilated precursor of HSP-inducing factor (thus activating it), or on its glycosilated inhibitor (thus disenabling its inhibitory potential, for example by preventing the formation of a complex with the factor), or both.

Modification or removal of glycosilic groups can be effected in the following ways:

1. By acid hydrolysis, using various acids or their derivatives.

2. By oxidative methods, using, among others, such chemical substances as periodates, permanganates, chromium, chromium 6, osmium, osmium tetrooxide, and their derivatives.

3. By oxidative enzymes, examplified for instance, by cytochromes and their derivatives.

4. By other enzymatic methods, using, among others, the following enzymes and their derivatives as stated by Table 1 below.

TABLE 1

| Enzyme category/type | Enzyme action/activity | |
|---|---|---|
| A. Endoglycosidaes, such as: | | |
| □-Amylase | Hydrolyzes a1,4-glucan bonds in polysaccharides with 3 or more | |
| 1,4-□D-Glucan glucanohydrolase | a1,4-bound glucose units | |
| EC3.2.1.1 | -Glc□1-96 4Glc= | |
| □-Amylase | Hydrolyzes a1,4-glucan bonds in polysaccharides with 3 or more | |
| 1,4-□-D-Glucan glucanohydrolase | a1,4-bound glucose units = | |
| EC3.2.1.1 | Glc□1-96 4Glc= | |
| Cellulase | Hydrolyzes □1,4-glucan bonds in □-glucans like cellulose = | |
| | Glc□1-96 4Glc= | |
| Chondrotinase ABC, | Catalyzes the release of chondrotin sulfate- and dermatan-sulfate-side | |
| protease-free | chain from proteoglycans | |
| Chondroitin ABC lyase EC 4.2.2.4 | $GalNo \text{-} a \text{-} {( \begin{matrix}chondroitinsulfate \\ dermatansulfate\end{matrix} )}_{n}$ | 9 GlcA-Gal-Gal-Xyl-Ser-Core protein |

| Endo-□-gatactosidase | Hydrolyzes internal □-galactosidic linkages of the lactosamine type | | |
|---|---|---|---|
| Keratan-sulfate 1,4-□-D-galactanohydrolase EC 3.2.1.103 | =GlcNAcB1 6 3GalB1 96 | $( \begin{matrix}3 \\ J\end{matrix} )$ | GlcNAc= |

| Endoglycosidase D | Active on N-linked oligosaccharides of glycopeptides/proteins. | |
|---|---|---|
| Endo-□-N-acetylglucosaminidase D | Cleaves only a MS high-mannose structure [x-Man□1 6 3(Man□166), | |
| EC 3.2.1.96 | y and z =H] and the core portion of a complex structure | |
| | after removal of most antennary residues with exoglycosidases | |
| | (x = H; y = h or GlcNAc; z = h or Fuc] | |
| | x-Man | |
| | Man 6 GlcNAc- 96 GlcNAc-Asn B peptide/protein | |
| | y-M\overline{a}nz | |
| Endoglycosidase F/N-Glycosidase F | Ψ see individual enzymes | |
| mixture of both glycosidases | | |
| EC 3.2.1.96 | | |
| Endoglycosidase F | Active on N-linked oligosaccharides of glycopeptides/proteins. | |
| Endo-□-N-acetylglucosaminidase | Cleaves only biantennary complex (x and y = AcNeuGalGlcNAc | |
| EC 3.2.1.96 | or similar, 2 = H or Fuc), to some extent high mannose type structures | |
| | (reaction rate is twenty fold lower) and biantennary hybrid type | |
| | glycans. | |
| | x-Man | |
| | Man 6 GlcNAc- 96 GlcNAc-Asn-peptide/protein | |
| | | * |
| | y-Man | z |

| Endoglycosidase H | Active on N-linked oligosaccharides of glycopeptides/proteins. | |
|---|---|---|
| Endo-□-N-acetylglucosaminidase H | Cleaves only high mannose structures [n = 2-150, x = (Man)1-2, y-H] | |
| EC 3.2.1.96 | and hybrid structures (n = 2, x and/or y = AcNeu-Gal-GlcNAc) | |
| | (Man)ₙ 6 Man | |
| | Man 6 GlcNAc-96GlcNAc-Asn-peptide/protein | |
| | x-Man | |
| | \overline{y} | |
| Endoglycosidase H | Active on N-linked oligosaccharides of glycopeptides/proteins. | |
| Endo-□-N-acetylglucosaminidase H | Cleaves only high mannose structures [n = 2-150, x = (Man)1-2, y-H] | |
| EC 3.2.1.96 | and hybrid structures (n = 2, x and/or y = AcNeu-Gal-GlcNAc) | |
| | (Man)ₙ 6 Man | |
| | Man 6 GlcNAc-96GlcNAc-Asn-peptide/protein | |
| | x-Man | |
| | \overline{y} | |
| N-Glycosidase A | Hydrolyzes all types of N-glycan chains from glycopeptides even those | |
| PNGase A | carrying □1,3-bound core fucose residues present in insect and plant | |
| EC 3.5.1.52 | glycoproteins | |
| | x-Man | |
| | Man 6 GluNAc-96GlcNAc-Asn-peptide/protein | |
| | | * |
| | x-M\overline{a}n | □ |
| | | 1 |
| | | 9 |
| | | 3 |
| | | Fuc |

| N-Glycosidase F | Hydrolyzes all types of N-glycan chains from glycopeptides and | | |
|---|---|---|---|
| PNGase F | glycoproteins unless they carry □-1-3 linked core fucose residues | | |
| Peptide-N-glycosidase F | present in insect and plant glycoproteins. Free of contaminating | | |
| Peptide-N⁴-(acetyl-□-glucosaminyl)- | proteolytic activities [x = H or sugar(s)] | | |
| asparagine amidase | x-Man | | |
| EC 3.5.1.52 | | | |
| | Man6GlcNAc6GlcNAc96-Asn-peptide/protein | | |
| | x-Man | | |
| H-Glycosidase F | Hydrolyzes all types of N-glycan chains from glycopeptides and | | |
| PNGase F | glycoproteins unless they carry □-1-3 linked core fucose residues | | |
| Peptide-N-glycosidase F | present in insect and plant glycoproteins. Free of contaminating | | |
| Peptide-N⁴-(acetyl-□-glucosaminyl)- | proteolytic activities [x = H or sugar(s)] | | |
| asparagine amidase | x-Man | | |
| EC 3.5.1.52 | | | |
| | Man6GlcNAc6GlcNAc96-Asn-peptide/protein | | |
| | x-M\overline{a}n | | |
| N-Glycosidase F | Hydrolyzes all types of N-glycan chains from glycopeptides and | | |
| PNGase F | glycoproteins unless they carry □-1-3 linked core fucose residues | | |
| Peptide-N-glycosidase F | present in insect and plant glycoproteins. Free of contaminating | | |
| Peptide-N⁴-(acetyl-□-glucosaminyl)- | proteolytic activities [x= H or sugar(s)] | | |
| asparagiine amidase | x-Man | | |
| BC 3.5.1.52 | | | |
| | Man6GlcNAc6GlcNAc96-Asn-peptide/protein | | |
| | x-M\overline{a}n | | |
| O-Glycosidase, BSA-free | Hydrolyzes GalB163GalNAc from O-glycans | | |
| Endo-□-N-acatygalactosaminidase | | | |
| O-Glycan peptide hydrolase | | Thr | |
| O-Glycopeptide endo-O-galac- | Gal□163GalNAc□1-96 | | -peptide/protein |
| Losyl-N-acetyl-a-galactosamino | | Ser | |
| hydrolase | | | |
| EC3.2.1.97 | | | |

| Lysozyme | Hydrolyzes GlcNAc□164 N-acetyl-muramic acid bonds of the | |
|---|---|---|
| Muramidase | polysaccharidide backbone of peptidoglycans | |
| Mucopeptide N-acetylmu- | =GlcA□67MurNAc□1964GlcAc□165MurNAc= | |
| ramoyl-hydrolase | | |
| EC 3.2.1.17 | | |
| B. Exoglycosidases, such as: | | |
| N-Acetyl-□-D-glucosaminidase | Exoglycosidase with broad aglycone specificity. In addition to B | |
| N-Acetyl-□-D-glucosaminide | acetyl-□-D-glucosaminides hydrolyzes N-acetyl-□-D- | |
| N-Acetylglucosaminohydrolase | galactosaminides at 20% of the reaction rate. | |
| EC 3.2.1.30 | GlcNAc | |
| | | □1-96X |
| | GalNAc | |

| N-Acetyl-□-D-glucosaminidase | Cleaves terminal N-acetylglucosamine residues which are □-linked | | |
|---|---|---|---|
| N-Acetyl-□-D-glucosaminide | to oligosaccharide, 4GlcNac, Man or pheno | | |
| N-acetylglucosaminohydrolase | GlcNAc□196X | | |
| EC 3.2.1.30 | | | |
| Amyloglucosidase | Cleaves terminal glucoses which are □-1,4-or □1,6-linked to an | | |
| 1,4-□-D-Glucan glucohydrolase | oligo- or polysaccharide of multiple glucose units. The product is | | |
| EC 3.2.1.3 | D-glucose. | | |
| | | 4 | |
| | =Glc□1-96 | | Glc□1 6 X |
| | | 6 | |
| Amyloglucosidase | Cleaves terminal glucoses which are □-1,4-or □1,6-linked to an | | |
| 1,4-□-D-Glucan glucohydrolase | oligo- or polysaceharide of multiple glucose units. The product is | | |
| EC 3.2.1.3 | D-glucose. | | |
| | | 4 | |
| | =Glc□1-96 | | Glc□1 6 X |
| | | 6 | |
| Amyloglucosidase | Cleaves terminal glucoses which are □-1,4-or □1,6-linked to an | | |
| l,4-□-D-Glucan glucohydrolase | oligo- or polysaccharide of multiple glucose units. The product is | | |
| EC 3.2.1.3 | D-glucose. | | |
| | | 4 | |
| | =Glc□1-96 | | Glc□1 6 X |
| | | 6 | |

| □-Fructosidase | Cleaves terminal frustose residues which are □2,1-linked in | |
|---|---|---|
| Invertase, Saccharase | □-frustofuranosides; x may be glucose or an oligosaccharide. | |
| □-D-Fructofuranoside | XI-962□fru | |
| fructohydrolase | | |
| EC 3.2.1.26 | | |
| □-1-Fucosidase | Cleaves terminal fucose residues which are a-linked to | |
| □-L-Fucoside fucohydrolase | oligosaccharides, glycopeptides, glycolipids or phenol. | |
| EC 3.2.1.51 | Rate of hydrolysis decreases rapidly with increasing size and | |
| | complexity of substrate. | |
| | Fuc□1-96X | |
| □-Galactosidase | Cleaves terminal galactose residues which are a-linked to | |
| □-D-Galactoside galactohydrolase | monosaccharide, oligosaccharide, glycopeptide or phenol | |
| EC 3.2.1.22 | Gal□1-96X | |
| □-Galactosidase | Cleaves terminal galactose residues which are □1,3-, □1,4- or | |
| □-D-Galactoside galactohydrolase | □1,6-linked to a monosaccharide, oligosaccharide, glycopeptides | |
| EC 3.2.1.23 | or glycolipid. Relative rate of cleavage is □163∃□164> | |
| | □166, determined on Gal6GlcNAc bonds in disaccharides. | |
| | Cleaves □1,6-linkages very slowly | |
| | Gal□1-_X | |
| □-Galactosidase | Hydrolyzes terminal galactose residues which are □1,4-linked to | |
| □-D-Galactoside galactohydrolase | GlcNAc (100% reaction rate) or Glc (23% reaction rate) | |
| EC 3.2.1.23 | | |
| | | Glc |
| | Gal□1-964 | |
| | | GlcNAc |

| | |
|---|---|
| □-Galactosidase | Cleaves terminal galactose residues which are □1,4-linked to a |
| □-D-Galactoside galactohydrolase | monosaccharide, oligosaccharide or glycopeptide |
| EC 3.2.1.23 | Gal□1-96X |
| □-Galactosidase | Especially used for labeling in the field of enzyme immunoassay |
| □-D-Galactoside galactohydrolase | techniques. |
| EC 3.2.1.23 | |
| □-Glucosidase (Maltase) | Cleaves terminal glucoses which are a-linked to mono-, oligo- or |
| □-Glucosidase glucohydrolase | polysaccharides or phenol. Prefers □1,4,-linkages, but will also |
| EC 3.2.1.20 | hydrolyze □1,2- and □1,3-linkages; hydrolyzes □1-6 linkages very |
| | slowly. Rate of hydrolysis decreases rapidly with increasing size |
| | and complexity of the substrate. |
| | Glc□1-96X |
| □-Glucuronidase | Cleaves terminal glucose acid which is □-linked to mono-, oligo |
| □-D-Glucoronide glucuronoso- | or polysaccharides phenol |
| Hydrolase | GlcA□1-96X |
| EC 3.2.1.31 | |
| □-Mannosidase | Broad aglycone specificity hydrolyzing Man□162 man, man □166 |
| □-D-Mannoside mannohydrolase | Man at 100%, and Man□163 Man at 7% reaction rate |
| EC 3.2.1.24 | |
| | Man□1-96X |
| Neuraminidase (Sialidase) | Cleaves terminal sialic acid residues which are □-2,3-, □2,6- or |
| Acyineuraminyl hydrolase | □2,8-linked to Gal, GlcNAc, GalNAc, AcNeu, GlcNeu, |
| EC 3.2.1.18 | oligosaccharides, glycolipids or glycoproteins. Relative rate |
| | of cleavage is □266 > □263 > □2 determined on bonds |
| | in tri- and tetrasaccharides. |
| | AcNeu□2-96X or GlcNeu□2-96X |
| C. Glycosyltransferases, such as: | |
| Galactosyltransferase | Transfers galactose residues from UDP-galactose to the C₄— |
| Lactose synthase | hydroxyl of N-acetylglucosamine in glycoproteins. Catalyzes the |
| UDPgalactose: D-glucose | synthesis of lactose in combination with a-lactalbumin under |
| 4-B-D-galactosyltransferase | formation of a □164 linkage |
| EC 2.4.1.22 | |
| UDPgalactose: N-acetyl-D- | |
| Glycosaminyl-glycopeptide | |
| 4-□-D-galactosyltransferase | |
| EC 2.4.1.90 | |
| 2,6-Sialyttransferase | Transfers sialic acid residues to the 6-position of the |
| CMP-N-acetyineuraminate: | Gal□164GlcNAc unit of glycoconjugates. The preparation is |
| □-D-galactodyl-1,4-N-acetyl- | used for in-vitro sialylation of Gal□(1-4)GlcNAc structures in |
| □-D-glucosamine a-2-6-N-acetyl- | N-glycans via a(2-6) links |
| neuraminyltransferase | |
| EC 2.4.99.1 | |

Oxidation Reaction Schemes

The general oxidative reaction for converting the precursor form entity into the activated Hsp-inducing factor is given broadly by Reaction 1 below.

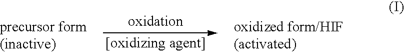

A more specified statement of conversion is given by reactions II, III and IV given below.

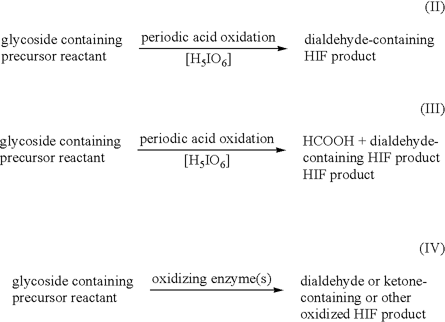

The oxidizing agents of choice presently include sodium periodate, periodic acid, and any other conventionally known chemical oxidizing agents. These are presently preferred for both in-vitro and in-vivo use. It is recognized that enzymatic oxidative conversion of the precursor to the dialdehyde form, or a ketone-containing form, or an otherwise oxidized form, is equally preferred and desirable.

For in-vivo or ex-vivo conversions, all conventionally known oxidizing agents capable of converting a glycoside-containing precursor entity into a dialdehyde-containing a ketone-containing entity, or an otherwise oxidized active factor are deemed to be within the scope of the present invention. Alternatively, the same scheme applies, in cases of deglycosilation, and thus deactivation of an inhibitor of the HIF.

In general, any agents capable of modification or removal of glycosilic groups or any other groups attached to proteins via glycosilic groups are deemed to be within the scope of the present invention. Thus, this change(s) can be effected by various acids and their derivatives; by oxidative chemical agents exemplified by periodates, permanganates, chromium and osmium tetraoxide and their derivatives; by oxidative enzymes examplified by cytochromes and their derivatives; and by endoglycosidases, exoglycosidases, and glycotransferases (examples of which are listed above) and their derivatives. Also it is understood that other agents (such as an antibody or antibody derivative having any of the activities described herein) also constitute suitable agents for use.

### III. Therapeutic Applications For HIF

(a) The expression of Hsp70 and other HSPs can be induced for therapeutic purposes in-vivo by administering a modifying treatment (such as sodium periodate or other oxidizing or deglycosilating treatment) which converts the inactive precursor into active factor or inactivates an inhibitor of the factor; by administering a modifying agent or factor; by stimulating the secretion of modifying activity (or its cofactor) by using agonists of the factor=s receptor; or by interfering with (activating) its signal transduction pathway. Therapeutic applications including but not limited to treatments of heart attack, stroke (ischemias), autoimmune diseases, neurodegenerative disorders and other conditions caused by cell death (apoptosis and necrosis).

(b) The HIF entity is demonstrably capable of inducing HSPs systemically, including the expression of Hsp70 and other HSP species in aged human cells incapable of a substantial expression of Hsp70 in response to stresses. Moreover, as shown, at certain concentrations ranges the factor does not induce Hsp70 in aged human cells but enables them to express Hsp70 in response to stresses. These findings constitute the basis for antiaging treatment where active factor is generated in-vivo (in aging subjects) in the desired concentration range by means outlined in the preceding paragraph. This will enable aged cells to express Hsp70 and other HSPs in response to stresses and thus restore stress resistance of aging subjects.

### VI. Experimental And Empirical Data

To demonstrate the merits and value of the present invention, a series of planned experiments and empirical data are presented below. It will be expressly understood, however, that the experiments described and the results provided are merely the best evidence of the subject matter as a whole which is the invention; and that the empirical data, while limited in content, is merely illustrative of the scope of the invention envisioned and claimed.

### Experimental Series I¹

Objectives

Stress-inducible Hsp72 expression, which is responsible for acquired stress tolerance, represents one of the major cellular protective systems. However, this line of defense is being progressively weakened and lost with aging which is characterized by a progressive impairment in the ability to adapt to environmental changes. Manifestation of this impairment at the cellular level is the age-related attenuation of inducibility of Hsp72 and the consequent loss of the ability to develop acquired stress tolerance. ¹Reproduced in part from Volloch, V. and S. Rits, Exp. Cell Res. 253: 483-492 (1999).

Attenuation of Hsp72 expression was shown in cells isolated from young organisms and aged in culture, in cells isolated from aged human donors and in aging animal model systems. It should be noted that the intrinsic sensitivity of cells to stresses does not change during aging; what changes is the ability to develop the acquired tolerance, because of attenuation of Hsp72 expression.

Age-related attenuation of Hsp72 expression results in increased stress sensitivity. It would be of significant importance to be able to activate Hsp72 synthesis in aged cells. The ability to manipulate the expression of Hsp72 in old cells could serve as a basis for developing effective therapies for treating age-related diseases such as ischemia and neurodegenerative disorders, where lack of inducible Hsp72 expression in response to damage is an important factor in pathology. Previously it has been demonstrated [Volloch et al., Cell Stress Chaperones 3: 265-271 (1998)] that old cells retain the capability to express Hsp72, and that age-related attenuation of Hsp72 expression and consequent stress sensitivity can be bypassed by treatment with a proteosome inhibitor (MG132) which induces Hsp72 synthesis via an alternative heat shock transcription factor HSF2. Treatment with a proteosome inhibitor led to induction of Hsp72, suppression of stress-induced JNK activation, and restoration of stress resistance. Unfortunately, MG132 is a highly toxic compound and cannot be used therapeutically.

In the search for an activator of Hsp72 expression the point of departure were the observations made by Davidson and co-workers with CH1 cells [Davidson et al., Mol. Cell. Biol. 15: 1071-1078 (1995)]. CH1 cells carry an unknown defect in heat shock response and do not express Hsp72 when subjected to elevated temperature. An addition of ascites fluid to cultures of CH1 cells restored their ability to express Hsp72 in response to heat shock. Davidson et al. concluded that mouse ascites fluid contains a diffusible agent that is able to compensate for the heat shock response defect of CH1 cells. In contradistinction, these experiments explored the alternative possibility of the ascites fluid containing a general activator of Hsp72. The experiments and data presented here demonstrate the detection of a nontoxic natural factor which is capable of activating a robust Hsp72 expression in both young and aged human fibroblasts that lost the ability to express Hsp72 in response to heat shock.

Methods and Materials

Cell Lines and Materials

Human fibroblasts IMR90 were grown in MEM supplemented with 20% fetal bovine serum and 2 mM glutamine. Murine lymphoma CHI cells were grown in RPMI medium supplemented with 10% fetal bovine serum and 50 □M mercaptoethanol. Rat myogenic cells H9c2 were grown in DME medium supplemented with 10% fetal bovine serum. Cells were seeded at a density 5×10⁴ cells/ml and grown at 37° C. in an atmosphere of 95% air and 5% CO₂. Cultures were replated when cell density reached confluence (8×10⁵/ml for CH1 cells). Experiments with IMR90 and H9c2 cells were carried out at approximately 70% confluence and with CH1 cells at 5×10⁵ cells/ml. Murine ascites fluids obtained from animals hosting hybridoma cells were a gift from Dr. Vic Raso (Boston Biomedical). Human samples were obtained from Massachusetts General Hospital. Since they are encoded, no data on the patients are known to us. SPA810 antibodies (specific for Hsp72), SPA 820 antibodies (specific for Hsp73 and Hsp72), and anti-Hsp40 antibodies were purchased from StressGen Biotechnologies. Antibodies against phosphorylated JNK were obtained from New England Biolabs.

Adenovirus-Based Expression of Antisense Hsp72 RNA

Recombinant adenovirus vector expressing antisense Hsp72 RNA was constructed by cloning a dicistronic transcription unit encoding Hsp72 in inverted orientation and Aequorea Victoria green fluorescent protein gene, separated by the encephalomyocarditis virus internal ribosome entry site, into an adenovirus transfer vector. Expression of this transcription unit is controlled by the tetracycline-regulated transactivator protein tTA which was expressed from a separate recombinant adenovirus. Adenovirus constructs were a gift of Dick Mosser, Biotechnology Research Institute, Montreal. Viruses were used at a stock concentration of 10¹⁰ PFU/ml. Infection of cells with both viruses simultaneously led to suppression of Hsp72 induction in the absence of tetracycline but not in the presence of 50 nM anhydrotetracycline. Two-milliliter cell cultures grown in 35-mm dishes were infected in the presence or the absence of anhydrotetracycline with 3×10⁷ PFU of each virus (60-100 PFU/cell; aged IMR90 cells are larger than young ones and therefore reach confluence at a smaller density), sufficient for infection of practically 100% cells as judged by the GFP expression. After a 12-h incubation with viruses, cells were washed with PBS and placed in fresh media with or without anhydrotetracycline.

Preparation and Analysis of Samples

IMR90 cells that underwent 66 population doublings (PDL 66) were lysed in a buffer containing 60 mM Tris-HCl, pH 7.4, 50 mM NaCl, 2 mM EDTA, 1% Triton X-100, 25 mM B-glycerophosphate, 10 mM NaF, 1 mM Na₃VO₄, and protease inhibitors (1 mM PMSF and 25 □g/ml each of aprotenin, pepstatin, leupeptin). Samples were subjected to SDS-PAGE followed by Western transfer to a nitrocellulose membrane and immunoblotting with antibodies against Hsp72, other HSPs, and phosphorylated (active) JNK. To ensure equal loading of gels, relative protein concentrations in samples were determined using Bio-Rad protein assay reagent, and loading volumes were adjusted so that equal amounts of protein were loaded. Moreover, equal loading was ascertained by Ponceau staining of membranes immediately following Western transfer. After incubation with antibodies and subsequent washing the membranes were incubated with secondary horseradish peroxidase-conjugated antibodies. Bands were visualized by ECL using ECL-Western blotting detection reagents kit from Amersham.

Quantitation of Stress-Induced Apoptosis

Cells were subjected to one of the following treatments: severe heat shock (45° C., 75 min), menadione (40 mg/ml in 2% serum), TNF (10 ng/ml in the presence of 10 □g/ml cyclohexemide), UV/C irradiation (400 J/m²), or etoposide (150 □g/ml). The extent of apoptosis was determined by Hoechst staining [18] after 24 h (heat shock, menadione, etoposide), 40 h (UV), or 6 h (TNF). Hoechst 33342 reagent was obtained from Molecular Probes and used at 1 □g/ml in accordance with the manufacturer=s instructions. The extent of apoptosis was scored by microscopy.

Results

### Experiment 1

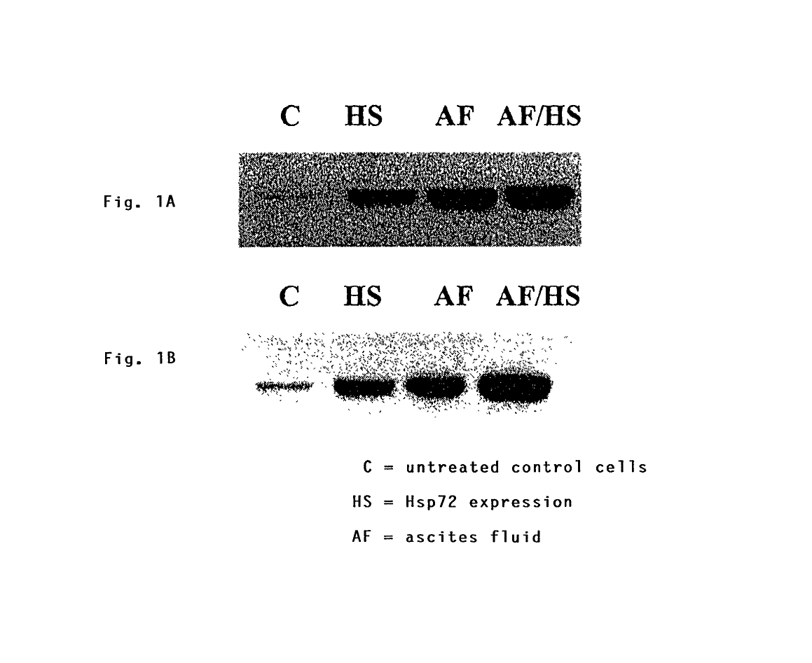

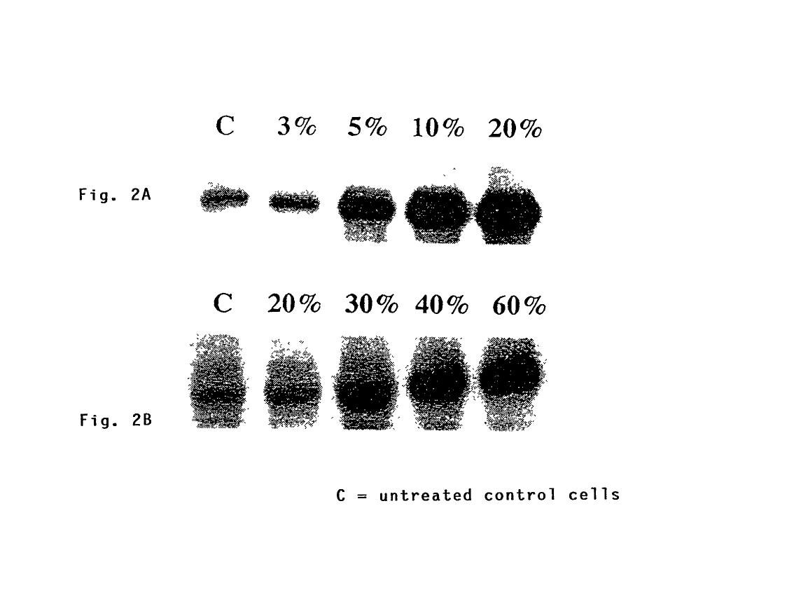

This experiment tested the hypothesis that ascites fluid might harbor an activator of Hsp72 expression. For this test, human fibroblasts IMR90 cells were used which underwent 36 population doublings (PDL 36) and H9c2, a rat myogenic cell line. These cells were incubated for 12 h in their regular culture media supplemented with 10% murine ascites fluid (AF). After such an incubation, cells were collected and Hsp72 levels were analyzed by SDS-PAGE followed by immunoblotting with SPA810 antibodies specific for Hsp72. The results are illustrated by FIGS. 1A and 1B respectively.

FIGS. 1A and 1B are immunoblottings of cells which demonstrate that AF (ascites fluid) induces Hsp72 expression in IMR90 fibroblasts and H9c2 rat myogenic cells; AF and heat shock have an additive effect on Hsp72 expression. IMR90 fibroblasts (PDL 36) of FIG. 1A and H9c2 rat myogenic cells of FIG. 1B were incubated in the presence or absence of 10% murine ascites fluid. After 12 h aliquots of cells were subjected to heat shock (44° C., 30 min) and incubated for an additional 8 h, and levels of Hsp72 were determined by immunoblotting.

As shown in FIG. 1, in both types of cells, incubation with AF induced Hsp72 expression at levels similar to those induced by heat shock (44° C., 30 min). Notably, the addition of AF to these cell lines was not toxic. In fact the addition of AF to cultures of IMR90 and H9c2 cells actually accelerated cell growth by approximately 25%, indicating that treatment with AF is not a stressful condition. When IMR90 and H9c2 cells were subjected to heat shock in the presence of AF, an additive effect was observed (FIG. 1); i.e., levels of Hsp72 expressed in response to AF and heat shock appeared to be equal to the sum of Hsp72 levels induced by AF alone and by heat shock alone.

### Experiment 2

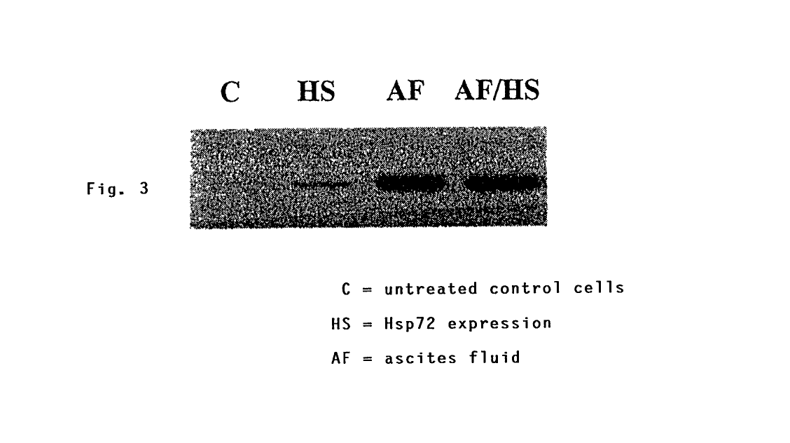

Six batches of murine AF were used as generated by different hybridomas as were 14 batches of human AF from patients with different forms of cancer (ovarian cancer, breast carcinoma, gastric carcinoma, and lung cancer). These sample batches were then evaluated for Hsp72 expression activity. The results are illustrated by FIGS. 2A and 2B respectively.

FIG. 2 shows that human and murine AFs have similar effects on Hsp72 expression albeit at different saturating concentrations. IMR90 fibroblasts (PDL 36) were incubated in the presence or absence of different concentrations of murine (FIG. 2A) or human (FIG. 2B) ascites fluid. After 12 h levels of Hsp72 were determined by immunoblotting.

Whereas there were minor differences in Hsp72-induced potential within murine or human batches, there was a significant difference between murine and human samples. In general, saturating concentrations of murine AF varied between 10 and 20%, and that of human AF between 40 and 60% (FIG. 2). At these concentrations, human and murine AFs had similar effects on Hsp72 expression.

### Experiment 3

The additive effect of AF and mild heat shock on Hsp72 expression observed in experiments with IMR90 and H9c2 cells suggested that AF may induce synthesis of Hsp72 through a pathway different from a heat shock response pathway. To test this possibility murine lymphoma CH1 cells were used. CH1 cells carry an unknown defect in their heat shock response pathway and do not synthesize Hsp72 when subjected to elevated temperatures. The results are illustrated by FIG. 3.

FIG. 3 demonstrates that AF induces Hsp72 expression in murine lymphoma CH1 cells through a pathway distinct from the heat shock response pathway. CH1 cells were incubated in the presence or absence of 10% murine ascites fluid. After 12 h aliquots of cells were subjected to heat shock (43° C., 18 min) and incubated for an additional 8 h, and levels of Hsp72 were determined by immunoblotting (the major inducible member of murine Hsp70 family is in fact 68-kDa protein and therefore should be called Hsp68; to simplify our terminology we refer to it as Hsp72).

When CH1 cells were subjected to mild heat shock (43° C., 18 min), only very unsubstantial Hsp72 expression was seen (FIG. 3). In contrast, when CH1 cells were incubated for 12 h in their regular culture media supplemented with 10% murine AF, a robust expression of Hsp72 was observed (FIG. 3). Importantly, when heat shock (43° C., 18 min) was administered in the presence of AF, little, if any, difference in levels of Hsp72 could be seen in samples with AF alone or with AF plus heat shock (FIG. 3). Therefore, it appears that induction of Hsp72 expression by AF occurs via a pathway distinct from the heat shock response pathway. The ability of the AF factor to induce Hsp72 expression in IMR90, H9c2, and CHI cells suggests that the AF factor is probably a general, rather than a cell-type specific, inducer of Hsp72 expression.

### Experiment 4

As mentioned above, the ability of aged IMR90 cells to express Hsp72 in response to heat shock is greatly diminished. The observation that the AF factor induces Hsp72 expression through a pathway distinct from the heat shock response pathway opened the possibility that it may induce Hsp72 expression in old cells that no longer respond to heat shock and other stressful treatments. To test this possibility, the test utilized young IMR90 cells that underwent 24 population doublings (PDL 24) and aged IMR90 cells that underwent 66 population doublings (PDL 66) with mild heat shock (44° C., 30 min) alone, with AF alone, or a combination of heat shock and AF.

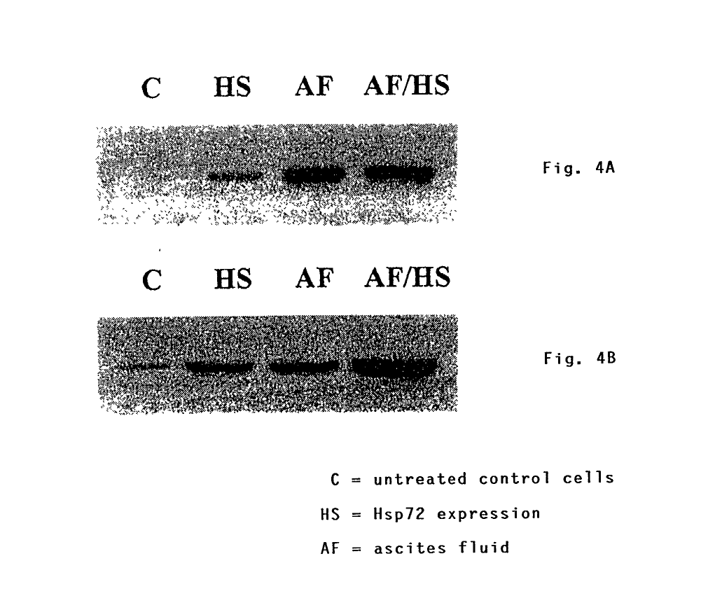

The results are illustrated by FIGS. 4A and 4B respectively.

FIGS. 4A and 4B demonstrate that AF induces Hsp72 expression in aged IMR90 cells that largely lost the ability to express Hsp72 in response to heat shock. Aged IMR90 cells (PDL 66) (FIG. 4A) and young cells (PDL 24) (FIG. 4B) were incubated in the presence or absence of 10% murine ascites fluid. After 12 h aliquots of cells were subjected to heat shock (44° C., 30 min) and incubated for an additional 8 h, and levels of Hsp72 were determined by immunoblotting.

Thus, whereas heat treatment elicited Hsp72 expression in young cells, very little Hsp72 was produced in aged cells in response to heat shock alone (FIG. 4). Remarkably, AF induced strong Hsp72 expression not only in young but also in aged IMR90 cells (FIG. 4). When a combination of heat shock and AF was applied, an additive effect on Hsp72 expression was observed in young IMR90 cells. In contrast, in aged cells the level of Hsp72 expression was no higher than that seen with AF alone (FIG. 4). These findings constitute an additional indication that AF and heat shock induce Hsp72 expression via distinct pathways.

### Experiment 5

To assess whether the AF factor has a specificity for Hsp72, immunoblotted samples obtained in Experiment 4 described above were combined with an antibody against Hsp40. Hsp40 is expressed in unstressed cells at a substantial level which is approximately doubled following heat shock. The results are shown by FIGS. SA and SB respectively.

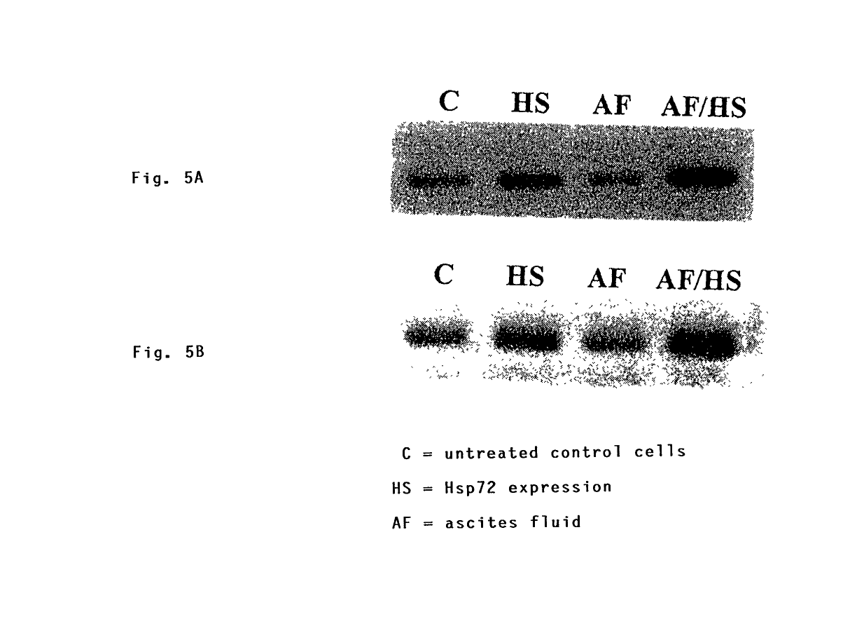

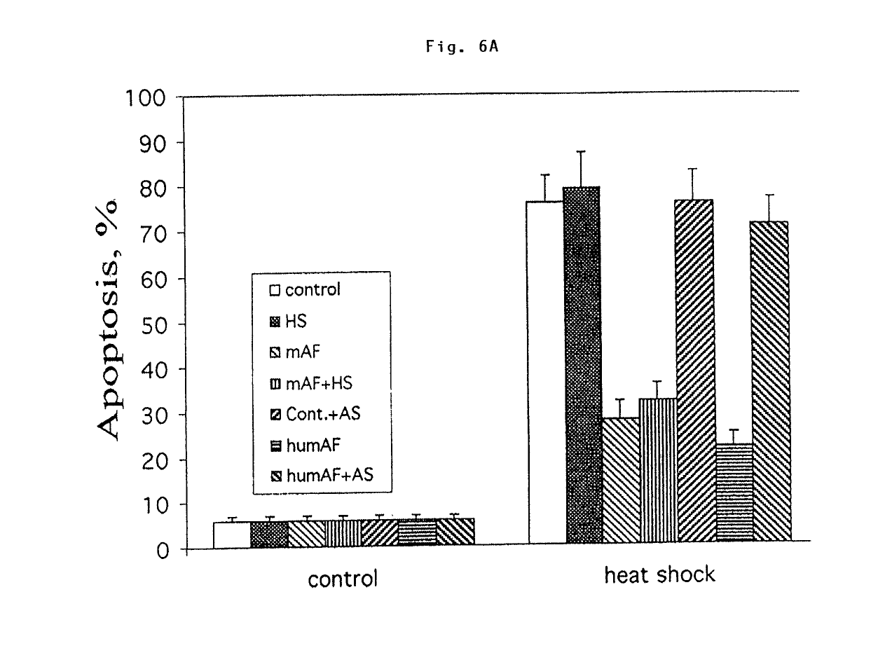

FIG. 5 as a whole demonstrates that AF does not induce expression of Hsp40 in either old or young IMR90 cells but potentiates the expression of Hsp40 by both types of cells in response to stress; heat-induced induction of Hsp40 is not attenuated in aged cells. Aged IMR90 cells (PDL 66) (FIG. SA) and young cells (PDL 24) (FIG. 5B) were incubated in the presence or absence of 10% murine ascites fluid. After 12 h aliquots of cells were subjected to heat shock (44° C., 30 min) and incubated for an additional 8 h, and levels of Hsp40 were determined in heat-treated and non-treated cells (in fact blots used in FIG. 4 were washed and analyzed with antibody against Hsp40).

Thus, as shown in FIG. 5, incubation with AF did not induce Hsp40 in either old or young IMR90 cells. Thus, this result suggests that the AF factor is not a general inducer of HSP but has some specificity for Hsp72. It also provides an additional indication that treatment with AF is not a stressful condition. Interestingly, although AF induced Hsp40 neither in young nor in aged IMR90 cells, it potentiated the expression of Hsp40 in response to stress in both types of cells (FIG. 5).

Also surprisingly, whereas aged cells exhibit attenuated expression of Hsp72 in response to heat shock (FIG. 4), the level of heat shock-induced Hsp40 in aged cells is very similar to that seen in young IMR90 cells (FIG. 5). This result indicates that age-related attenuation of HSP expression may be differentially regulated.

### Experiment 6

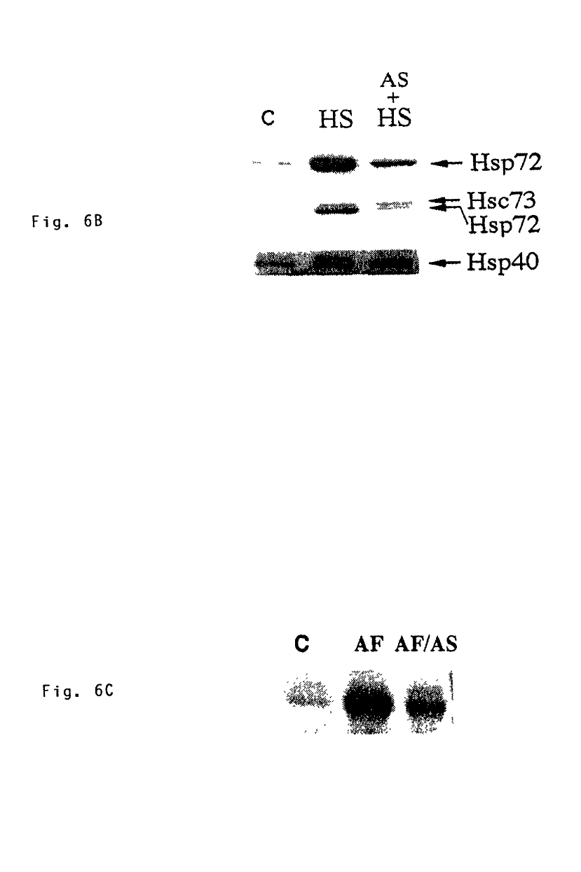

Since AF induces Hsp72, it was plausible that incubation of aged IMR90 cells with AF will protect them from apoptosis induced by otherwise lethal heat shock, similar to that seen when Hsp72 was expressed in aged IMR90 cells from an adenoviral construct. To test this possibility, aged IMR90 cells (PDL 66) were pretreated with mild heat shock (44° C., 30 min, a stress regimen well below the apoptotic threshold for IMR90 cells) alone, murine AF alone, or a combination of AF and mild heat shock. The results are illustrated by FIGS. 6A, 6B and 6C respectively.

FIG. 6 as a whole reveals that AF treatment protects old cells from apoptosis induced by otherwise lethal heat shock; Hsp72 is essential for the protective effect of AF. FIG. 6A illustrates the IMR90 cells, PDL 66, which were pretreated by mild heat shock (44° C., 30 min, well below the apoptotic threshold for IMR90 cells) alone, by 10% murine AF alone, or by a combination of AF and mild heat shock. Twelve hours later pretreated cells as well as nonpretreated cells were subjected to severe heat shock (45° C., 75 min) and the extent of apoptosis was determined 24 h after heat shock by Hoechst staining. To assess the role of Hsp72, cells (PDL 66) were infected with adenoviral constructs expressing antisense Hsp72 RNA under the control of tetracycline-inhibitable transactivator and maintained in the presence (to prevent expression of antisense RNA) or in the absence (to allow Hsp72 antisense RNA synthesis) of tetracycline. Cells were treated 72 h later with human ascites fluid (50%) for 12 h and subjected to severe heat shock (45° C., 75 min). The extent of apoptosis was determined 24 h after heat shock by Hoechst staining. mAF, murine AF; humAF, human AF. Each point represents the mean and standard deviation of triplicate determinations.

FIG. 6B shows that antisense Hsp72 RNA specifically suppresses the expression of Hsp72 but not of other HSPs. IMR cells (PDL 36) were infected with adenoviral constructs expressing antisense Hsp72 RNA and maintained in the presence or in the absence of tetracycline. Cells were subjected 36 h later to mild heat shock (44° C., 30 min) and incubated for 16 h at 37° C., and levels of Hsp72, Hsp73 and Hsp40 were determined by immunoblotting with SPA810 antibodies specific for Hsp72, SPA820 antibodies specific for Hsp73 and Hsp72, and anti-Hsp40 antibodies.

FIG. 6C demonstrates that Hsp72 antisense RNA prevents accumulation of Hsp72 following treatment with AF. IMR cells (PDL 66) were infected with adenoviral constructs expressing antisense Hsp72 RNA and maintained in the presence or in the absence of tetracycline. After 72 h cells were treated with 50% human AF for 12 h and Hsp72 levels were analyzed by immunoblotting.

Overall therefore, these pretreatments did not elevate the basal level of apoptosis (FIG. 6A). Twelve hours later pretreated cells as well as nonpretreated cells were subjected to severe heat shock (45° C., 75 min). Massive apoptosis was observed in nonpretreated cells (FIG. 6A) 24 h after severe heat shock. Pretreating of aged cells did not induce synthesis of Hsp72 (FIG. 4), nor did it inhibit apoptosis (FIG. 6A). This is in drastic contrast with young cells where pretreatment with mild heat shock induces the expression of Hsp72 and protects from a subsequent severe heat shock. On the other hand, pretreatment of aged cells with AF resulted in substantial suppression of apoptosis following severe heat shock (FIG. 6A). Finally, preexposure to a combination of AF and mild heat shock caused no greater suppression of apoptosis than AF alone (FIG. 6A).

To assess the role of Hsp72 in protection from heat-induced apoptosis conferred to aged IMR90 cells (PDL 66) by pretreatment with AF, an adenoviral construct expressing antisense Hsp72 RNA under the control of a tetracycline-inhibitable transactivator was used. Three aliquots of cells to be subjected to heat shock were infected with adenoviral constructs as described above and maintained for the duration of the experiment in the presence or absence of tetracycline. One aliquot, maintained in the presence (to prevent expression of antisense RNA) of tetracycline, served as control. After 72 h two aliquots, one maintained in the presence and another in the absence (to allow Hsp72 antisense RNA synthesis) of tetracycline, were supplemented with 50% human AF. Twelve hours later samples from all three aliquots were taken to assess the effect of antisense RNA on AF-induced Hsp72 expression (FIG. 6C), and two AF-treated aliquots as well as an aliquot not treated with AF and maintained in the presence of tetracycline were subjected to severe heat shock (45° C., 75 min). As shown in FIG. 6A, human AF conferred to aged IMR90 cells strong protection from otherwise lethal heat shock. Expression of antisense Hsp72 RNA significantly reduced induction of Hsp72 by AF (FIG. 6C) and virtually abolished the protective effect of the AF factor (FIG. 6A). Three control aliquots, not subjected to heat shock, were also infected with adenoviral constructs. One was maintained in the presence of tetracycline. Another was treated with AF in the presence of tetracycline. A third aliquot was treated with AF and maintained in the absence of tetracycline. None of these treatments elevated the basal level of apoptosis.

In a parallel experiment a test was made to determine whether expression of antisense Hsp72 RNA specifically suppresses the expression of Hsp72 but not of other HSPs. For this purpose we used IMR90 cells, PDL 36, and a protocol similar to that described above except that instead of AF, mild heat shock (44° C., 30 min) was used (heat shock was used in order to induce Hsp40 which is not induced by AF; young cells were employed because heat shock does not induce Hsp72 in aged cells). As shown in FIG. 6B, expression of antisense Hsp72 RNA specifically inhibits synthesis of Hsp72 and does not affect the expression of Hsp40 and even of closely related Hsp73. These results, taken together with the effect of Hsp72 antisense RNA on AF-conferred cytoprotection, strongly indicate that expression of Hsp72 is essential for the protective effect of the AF factor.

### Experiment 7

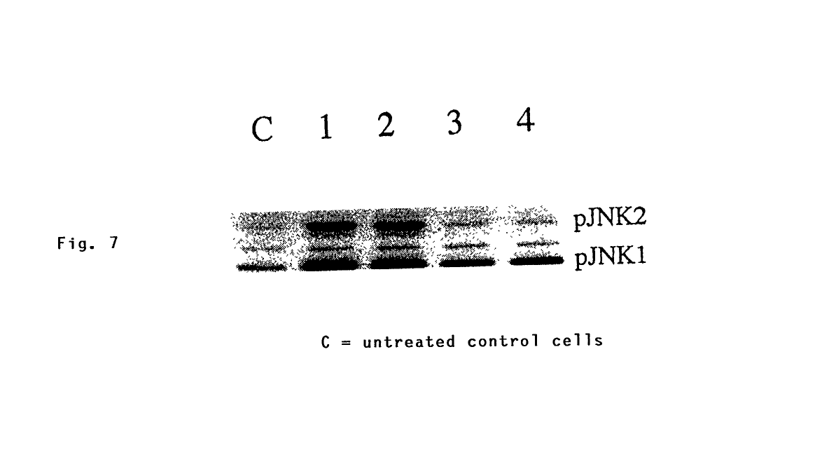

It was learned from previous work using aged IMR90 cells that Hsp72, when expressed from a viral construct, protects cells from heat-induced apoptosis by inhibiting activation of stress kinase JNK. Therefore, if AF protects cells through induction of Hsp72 expression, one should see suppression of heat-induced JNK activation in AF-treated cells. To test this prediction, aged IMR90 cells (PDL 66) were pretreated with mild heat shock (44° C., 30 min) alone, murine AF alone, or a combination of AF and mild heat shock. Twelve hours later pretreated cells as well as nonpretreated cells were subjected to severe heat shock (45° C., 75 min), and levels of active JNK were measured by immunoblotting with antibodies against phosphorylated (active) JNK. The results are illustrated by FIG. 7.

FIG. 7 demonstrates that AF treatment suppresses JNK activation in aged cells following severe heat shock. IMR90 cells, PDL 66, were pretreated by mild heat shock (44° C., 30 min) alone (2), by 10% murine AF alone (3), or a combination of AF and mild heat shock (4). Twelve hours later pretreated cells as well as nonpretreated cells (1) were subjected to a severe heat shock (45° C., 75 min). JNK activity was analyzed with antibody against phosphorylated (active) JNK.

As shown in FIG. 7, heat shock strongly activated JNK in nonpretreated cells. Pretreatment with mild heat shock (which did not induce Hsp72) did not inhibit JNK activation following severe heat shock. In contrast, in AF-treated cells (in which Hsp72 accumulated) heat-induced JNK activation was strongly suppressed. Pretreatment with a combination of AF and mild heat shock caused no greater suppression of apoptosis than pretreatment with AF alone. These results are consistent with the notion that Hsp72-inducing AF factor confers cytoprotection through suppression of JNK activation.

### Experiment 8

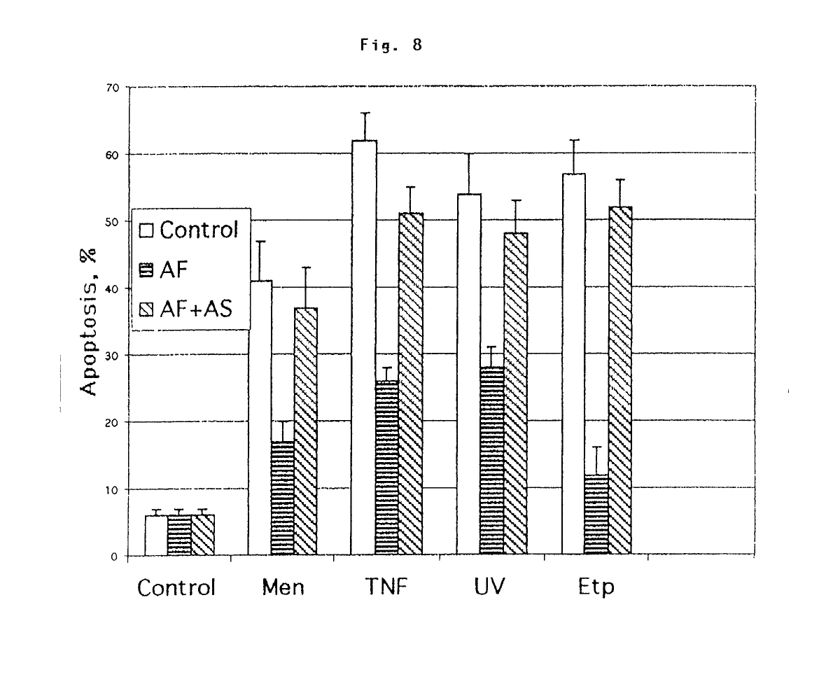

Two other important questions remained to be answered. Can the AF factor protect aged human cells from stresses other than heat shock and, if so, is Hsp72 expression essential for AF-conferred protection from other stresses? To address these questions, this experiment utilized adenoviral constructs expressing antisense Hsp72 RNA under the control of a tetracycline-inhibitable transactivator. Four sets of cultures (for four different stressful treatments) each consisting of three aliquots of cells were infected with adenoviral constructs as described above and maintained for the duration of the experiment in the presence or absence of tetracycline. After 72 h two aliquots in each set, one maintained in the presence and another in the absence of tetracycline, were supplemented with 50% human AF. Twelve hours later all three aliquots from each setXtwo AF-treated aliquots as well as an aliquot not treated with AF and maintained in the presence of tetracyclinexwere subjected to one of the following treatments: menadione, TNF, UV/C irradiation, or etoposide. The results are graphically illustrated by FIG. 8.

FIG. 8 reveals that AF protects aged cells from diverse stresses; the protection is due to AF-induced Hsp72 expression. IMR90 cells, PDL 66, were infected with adenoviral constructs expressing antisense Hsp72 RNA under the control of tetracycline-inhibitable transactivator and maintained in the presence or in the absence of tetracycline. After 72 h human AF was added to cell cultures to 50%. Twelve hours later cells were subjected to one of the following treatments: menadione (40 □g/ml in 2% serum), TNF (10 ng/ml in the presence of 10 □g/ml cyclohexemide), UV/C irradiation (400 J/m2), or etoposide (150 □g/ml). The extent of apoptosis was determined by Hoechst staining after 24 h (menadione, etoposide), 40 h (UV), or 6 h (TNF). Control cells were also infected and maintained in the presence of tetracycline. Each point represents the mean and standard deviation of triplicate determinations.

Accordingly, the data of FIG. 8 shows that preexposure to AF protected old cells from all of these treatments but expression of antisense Hsp72 RNA dramatically reduced the protective effect of AF factor with all stresses tested. Note also that three control aliquots, not subjected to stressful treatments, were also infected with adenoviral constructs. One was maintained in the presence of tetracycline, another was treated with AF in the presence of tetracycline, and a third aliquot was treated with AF and maintained in the absence of tetracycline. None of these treatments elevated the basal level of apoptosis. These results demonstrate that AF protects aged cells not only from heat shock but also from a variety of stresses of a different nature, and that expression of Hsp72 is essential for the protective effect of the AF factor. Considering the close link between oxidative damage and aging, it is both interesting and encouraging that AF conferred to old human cells protection from oxidative stress.

### Experiment 9

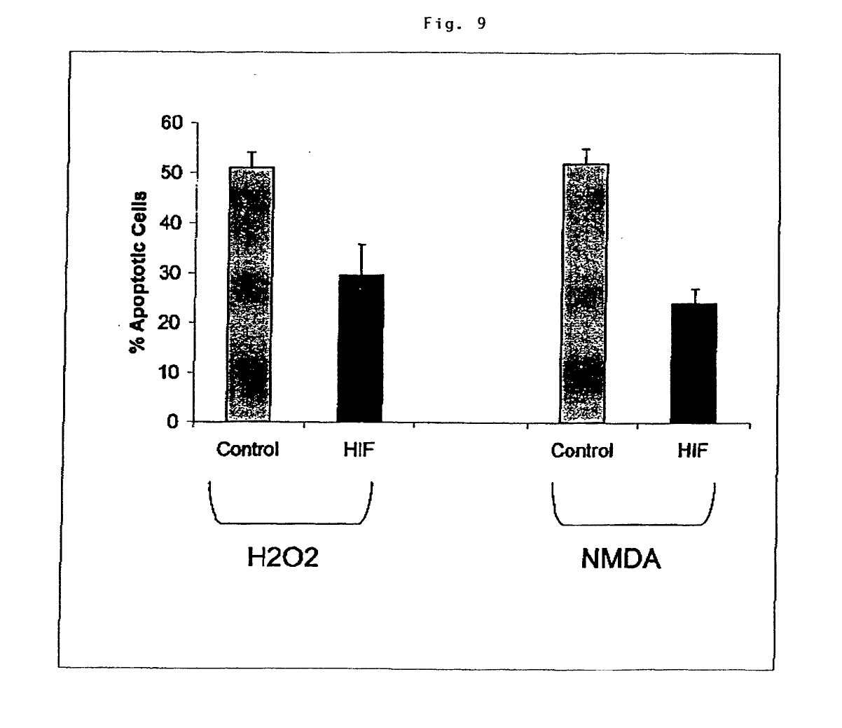

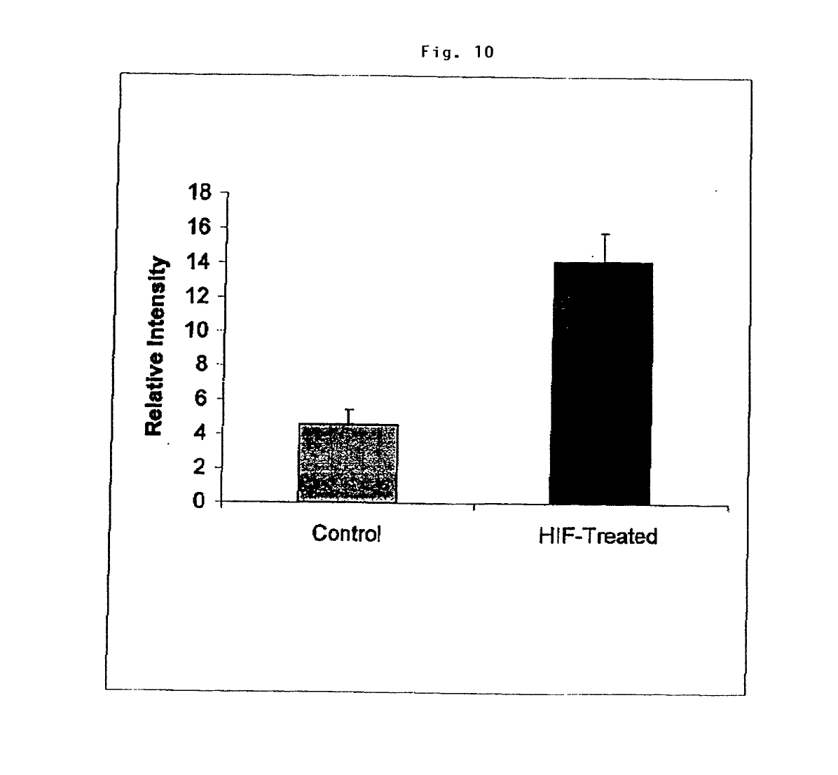

Because of the interest in stroke protection, one wanted to learn whether the HSP-inducing factor is capable to induce HSPs in neurons; and, if so, whether this would protect neurons from stresses. To evaluate these aspects, primary rat neuronal cultures were used. Cells were either untreated (control) or 20% AF was added to medium. After 24 hours aliquots were taken to measure the expression of HSPs and cultures were treated with either hydrogen peroxide or NMDA. Treatment with AF induced the expression of Hsp70 and Hsp27 in neurons and, as shown in FIG. 9, significantly decreased the extent of cell death (apoptosis) in cultures treated with either hydrogen peroxide or NMDA.

### Experiment 10

As shown in FIG. 10, this experiment demonstrates that the HIF induces the expression not only of Hsp70, but also of another cytoprotective protein, Hsp27. In this experiment, CH-1 cells were treated with murine AF and the expression of Hsp27 was analyzed as described above by polyacrylamide gel electrophoresis, western transfer and immunoblotting with Hsp27-specific antibody.

### Experiment 11

This experiment was designed to test the feasibility of antiaging treatment by the HIF. As was mentioned above, aged cells lose the ability to express Hsp70 in response to stresses, but treatment with the HIF results in the activation of Hsp70 expression in aged cells. On the other hand a prolonged expression of Hsp70 may lead to deleterious consequences [Volloch, V. and M. Sherman, Oncogene 18: 3648-3651 (1999)]. Accordingly, a possibility was tested that the HIF may potentiate Hsp70 expression in aged cells, i.e., enable aged cells to express Hsp70 in response to stresses.

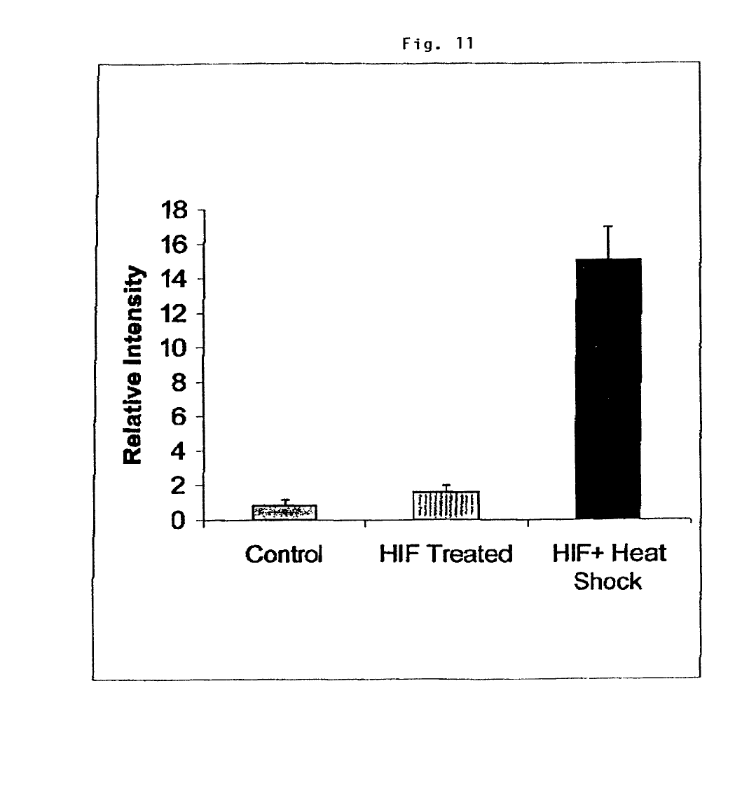

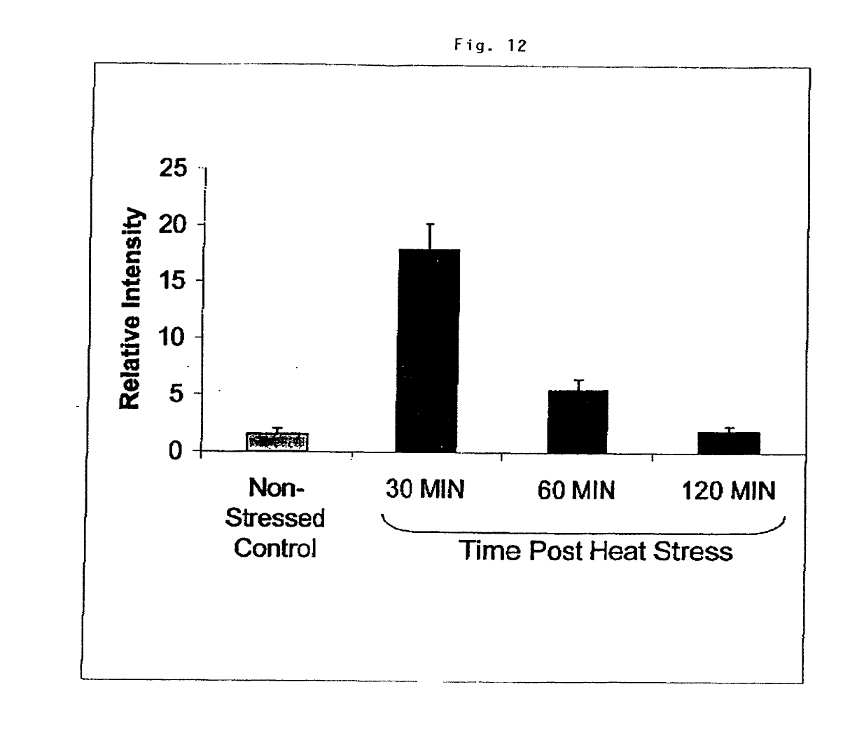

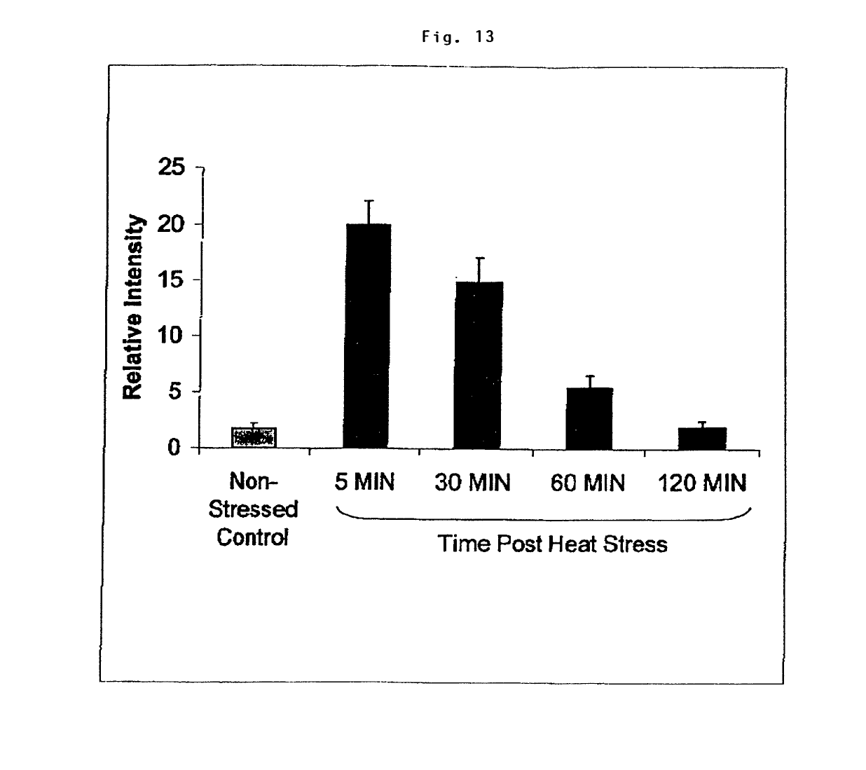

In this experiment aged IMR90 cells, PDL 66, that lost the ability to express Hsp70 in response to heat stress (44° C., 30 min.) were maintained in the presence of 3% of murine AF. This treatment alone did not result in the activation of Hsp70 expression. However, when cells maintained in the presence of 3% of murine AF were subjected to heat shock (44° C., 30 min.), vigorous Hsp70 expression at the levels comparable to those seen in stressed young cells was observed (FIG. 11).

These results lead to the conclusion that HIF at potentiating concentrations (sub-Hsp70 inducing concentration, i e., concentrations not inducing the expression of Hsp70 but enabling it in response to stresses in aged cells) can be used as an anti-aging therapeutic agent to restore stress response in aged human cells

### Experimental Series 2

Previous experiments addressed properties, potential and mechanisms of action of the HSP-inducing factor (HIF) which appears in ascites fluid in response to peritoneal tumor growth. However, this leaves open the question of the normal (non-tumor-related) role for the HIF. It is hypothesized that a normal physiological function of the HIF is that of a mediator of stress response. This hypothesis makes two verifiable predictions. First, that the active HIF would appear in blood serum in response to stresses; and second, that the HIF activity will disappear relatively rapidly following the cessation of a stress. These predictions as well as other properties and the potential of the HIF related to its normal physiological function were addressed in the following experiments.

### Experiment 12

To assess the physiological role of the HIF in-vivo, live C57 mice were subjected to a heat shock (42.7° C., 15 minutes). Prior to heat shock, all mice, including controls, were anaesthetized. Temperature was monitored by a rectal probe, and 1 ml of saline was injected subcutaneously (SQ) immediately after heat shock. Following heat shock, 0.1 ml aliquots of blood were collected at different time points from the tail of the same animals. Serum was prepared and added, at 15%, to cultures of CHI cells; after overnight incubation cells were collected, lysed, and analyzed for the expression of Hsp70 by SDS-PAGE followed by immunoblotting with Hsp70-specific antibody.

FIG. 12 reveals the results of this experiment; and shows that HSP-inducing activity was found to be present at high level in serum, in the very first sample, 30 minutes after heat shock. HIF level declined at 1 hour after heat shock and returned to a practically background level 2 hours after heat shock. These results indicate that (a) HSP-inducing activity indeed appears in blood soon after a stress; (b) it is unstable; and (c) it disappears relatively quickly after cessation of the stress, thus confirming all predictions made above.

### Experiment 13

To narrow the time window for the appearance of HSP-inducing activity following stress, a repeat of the prior heat shock experiment [experiment 12] was performed with rats starting with the collection of blood samples 5 minutes after heat shock. Serum was collected and analyzed for the presence of HSP-inducing activity using cultures of Rat-1 cells. The data, seen in FIG. 13, showed that the maximal level of HSP-inducing activity is seen, in the very first sample, collected 5 minutes following heat shock. A high level of HIF is sustained at 30 minutes after heat shock, declines at 1 hour and returns to a background level 2 hours after heat shock. Thus, these results support the conclusions drawn from the preceding experiment.

### Experiment 14

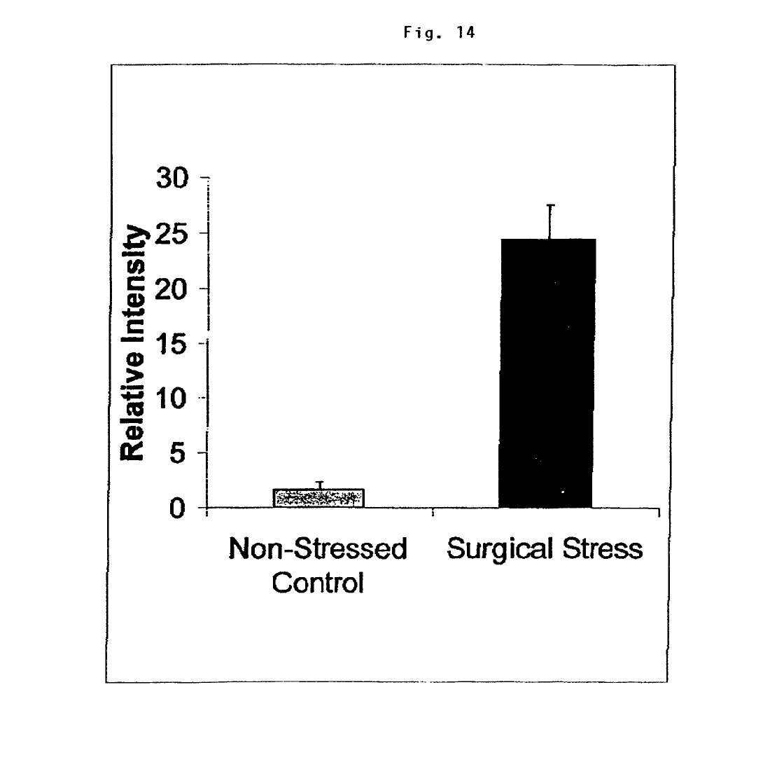

Probably the most interesting parameter observed in the heat shock experiments was the speed of the appearance of HSP-inducing activity in blood following stress. It is seen at the maximal level 5 minutes following heat shock, but this is in addition to 15 minutes of stress. To further narrow the window of the appearance of HSP-inducing activity, this experiment subjected mice to a different type of stress, surgical manipulation. Live mice were anaesthetized; the chest cavity quickly opened, the heart was punctured with a needle, and the blood collected into a syringe. The whole procedure lasted only 2 to 3 minutes. Serum was then prepared and analyzed for the presence of HSP-inducing activity using cultures of CH1 cells. The results, seen in FIG. 14, showed that this kind of stress led to the appearance in blood of HSP-inducing activity at the level comparable with that seen after heat shock.

The extremely fast appearance of HSP-inducing activity following a stress leads to a very important conclusion. It excludes a de novo synthesis of HSP-inducing factor following a stress; and suggests the following alternative possibilities. First, that active factor is present intracellularly and is secreted in response to stress. Second, that the factor is constitutively present in serum. In the latter case, its activity may be suppressed by an inhibitor; or it may be lacking an essential cofactor (secreted in response to stresses); or it may be present as inactive precursor, which is activated by a modifying (enabling) activity secreted in response to stresses.

### Experimental Series 3

In accordance with the observations described in the preceding experiments, the extremely fast appearance of HSP-inducing activity in blood of stressed animals could be explained by conversion of inactive precursor form of the HIF constitutively present in serum into active form-most likely by secretion of a modifying agent (or of its cofactor) that either modifies an inactive form of HIF and thus activates it, or modifies an inhibitor and thus suppresses it. If so, one expects that treatment of blood serum with a suitable agent will generate HSP-inducing activity. To test this view, in-vitro and in-vivo experiments were undertaken using sodium periodate as the modifying (oxidizing) agent.

### Experiment 15

Activation In-Vitro

a. Procedure

To activate HSP-inducing factor, 200 mM solution of sodium periodate in physicological saline (solutions of sodium periodate in water or in 50 mM sodium acetate pH 5.0 work equally well) was added to aliquots of serum or of ascites fluid to final concentration of 1 mM to 20 mM (at these concentrations and in conditions outlined below, sodium periodate oxidizes only sugar groups). Mixtures were incubated at room temperature in darkness for 1 hour. Following the incubation sodium periodate was quenched by the addition of solution of potassium borohydride in physiological saline to a final concentration equal to 1.5 times excess of the concentration of sodium periodate. The completeness of quenching was verified by using potassium iodide-starch test paper.

b. Assay

Aliquots of serum, treated as described above, were added to cultures of CH1 cells and cultures of primary human fibroblasts at 20% of final volume. Cultures were incubated for 16 hours at regular tissue culture conditions (37° C., 5% CO₂). Following the incubation, cells were collected, washed, and lysed. Lysates were subjected to polyacrylamide gel electrophoresis (PAGE), transferred to nitrocellulose membrane (Awestem transfer≅), and Hsp70 was detected by immunoblotting with Hsp70-specific monoclonal antibody (Spa810) and visualized by ECL technique.

c. Results

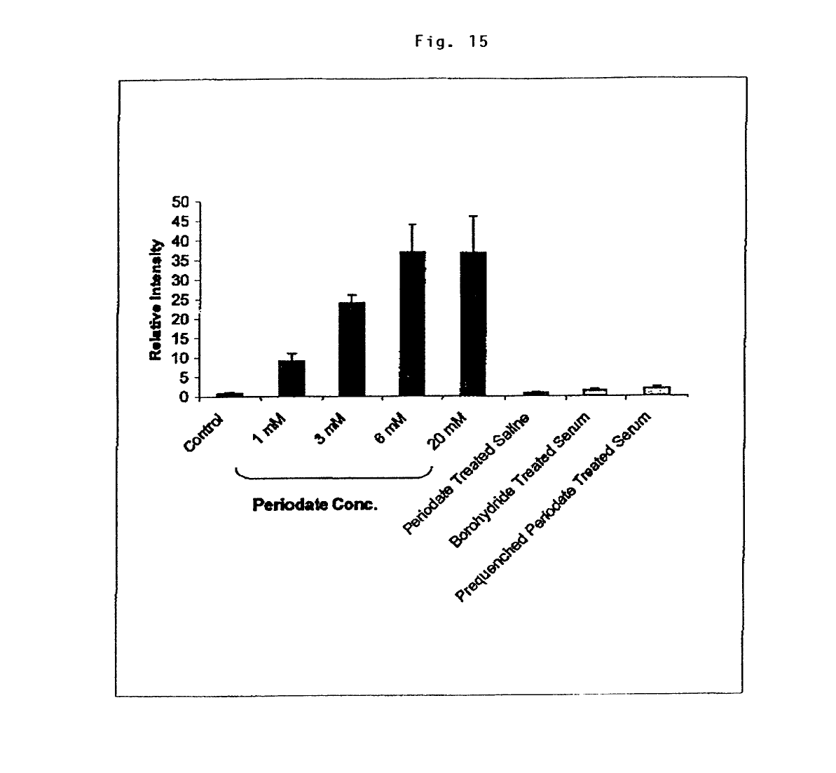

Treatment with sodium periodate strongly activated HSP-inducing factor in blood serum and in ascites fluid. Levels of active factor (assessed by the levels of Hsp70 in cell lysates) were directly proportional to concentrations of sodium periodate used. At maximal sodium periodate concentration levels, Hsp70 in cell lysate was at least 50 times that of its levels in cells incubated with untreated serum when serum was used. In control experiments where cells were incubated either with periodate-treated saline or with serum treated only with potassium borohydride, or with serum treated with prequenched periodate (periodate mixed with potassium borohydride as described above prior to the addition to serum), no activation of HSP-inducing factor was observed. Results of this experiment are illustrated in FIG. 15.

d. Interpretation

At concentrations used and in conditions outlined above, sodium periodate oxidizes only glycosilic groups. The results demonstrate therefore that inactive precursor (as defined above) of HSP-inducing factor is present in serum in a form of glycoprotein in substantial amounts and can be activated by oxidation or removal of glycosilic groups or by the removal of any other groups (for example lipids) attached to protein via glycosilic groups. Alternatively, HSP-inducing factor is present in serum in substantial amounts but its activity is suppressed by a glycosilated inhibitor; oxidation or removal of glycosilic groups inactivates the inhibitor and thus activates the HSP-inducing factor. In both scenarios oxidation or removal of glycosilic groups results in activation of otherwise inactive HSP-inducing factor.

### Experiment 16

Activation In-Vivo

a. Activation by Single Injection of Periodate

Mice were injected with either intraperitoneally (IP) or subcutaneously (SQ) with 0.2 ml of sodium periodate solution in physiological saline, or with 0.2 ml of physiological saline alone.

Four different concentrations of sodium periodate were used: 15 mM, 30 mM, 60 mM, and 120 mM. In parallel experiments the same concentrations of periodate but prequenched with potassium borohydride as described above were injected. After 2 hours blood samples were collected from tail and serum was used in the assay described above.

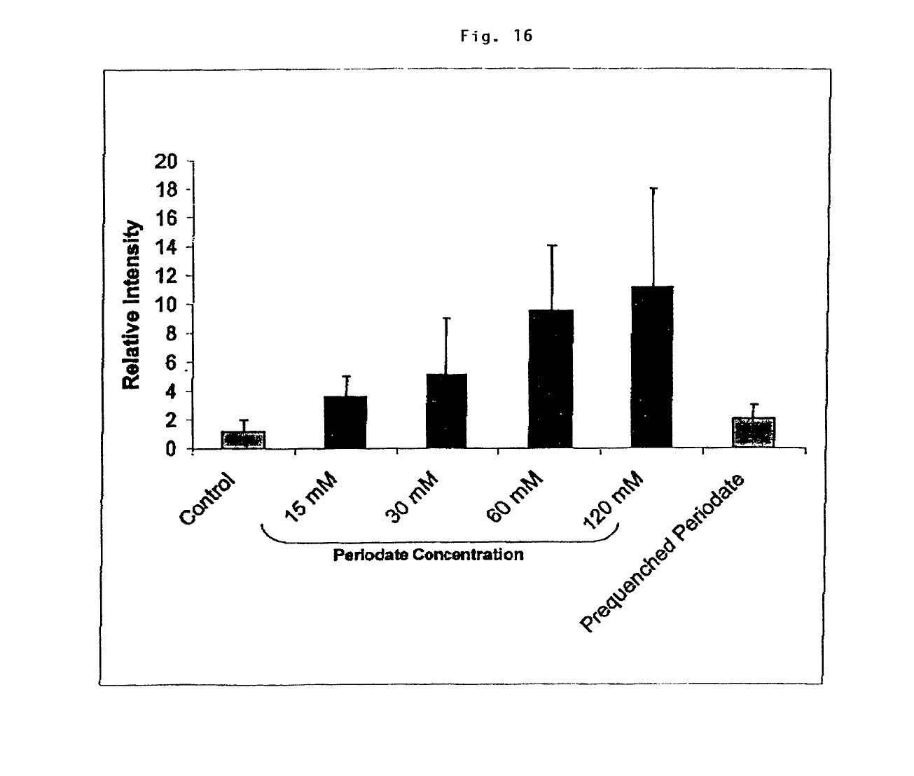

All serum samples from periodate-injected but not from saline-injected animals contained substantially increased levels of HSP-inducing factor. Levels of HSP-inducing factor were directly proportional to the concentrations of periodate used. At maximal sodium periodate concentration levels of HSP in cell lysates was about 10 times that of its levels in cells incubated with untreated serum. No increase in levels of HSP-inducing factor was seen when prequenched periodate was injected. Thus, the conclusion is that oxidation of glycosilic groups in serum in-vivo activates HSP-inducing factor by one of the mechanisms described above. Results of this experiment are illustrated in FIG. 16.

b. Activation by Prolonged Release of Periodate

In another type of experiment 110 01 of either 500 mM solution of sodium periodate in physiological saline or physiological saline alone were released over a period of 72 hours using microosmotic pumps implanted SQ. In parallel experiments the same concentration of periodate but prequenched with potassium borohydride as described above was administered. After different time periods blood samples were collected from tail and serum was used in the assay described above. Substantially increased levels of HSP-inducing factor were detected after 3 hours and maintained for the duration of the treatment in serum samples from periodate-administered but not from saline-administered or prequenched periodate-administered animals.

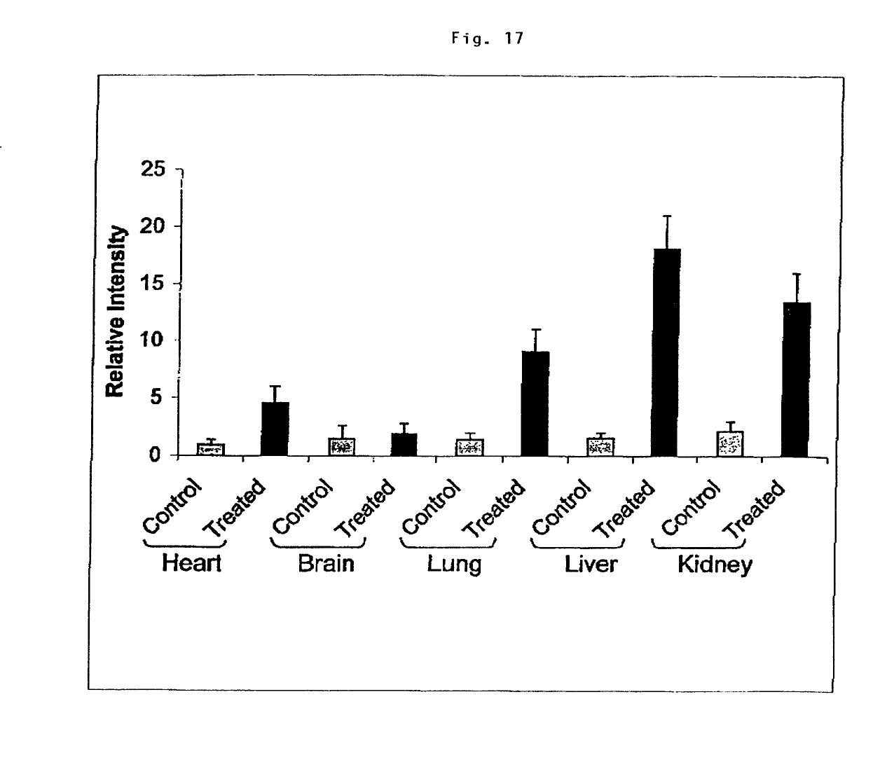

After 72 hours animals were sacrificed and different tissues were tested for the levels of Hsp70 as described above. As shown in FIG. 17, in all tissues tested (heart, lung, liver, kidney) with the exception of brain (probably because of blood-brain barrier) levels of Hsp70 were substantially (5 to 10 times) higher than in control, saline-administered or prequenched periodate-administered animals. Accordingly, the conclusion drawn is that prolonged administration of periodate or other deglycosilating agents results in increased levels of Hsp70 in tissues due to activation of HSP-inducing factor in blood serum.

### Points Of Emphasis

As given above herein, the results describe and experimentally evaluate HSP inducing factor in ascites fluid (AF), stress-activated HSP inducing factor in blood serum, periodate-activated HSP-inducing factor in stress serum and other physiological fluids, and HSP inducing factor in serum of periodate-treated mice. Several lines of evidence indicate that the heat shock protein inducing factor observed n all these different settings is one and the same.

Observations substantiating the notion that stress-induced and periodate treatment-generated HIF activity is the same, and is, in fact, identical to the AF-derived HIF, include the following:

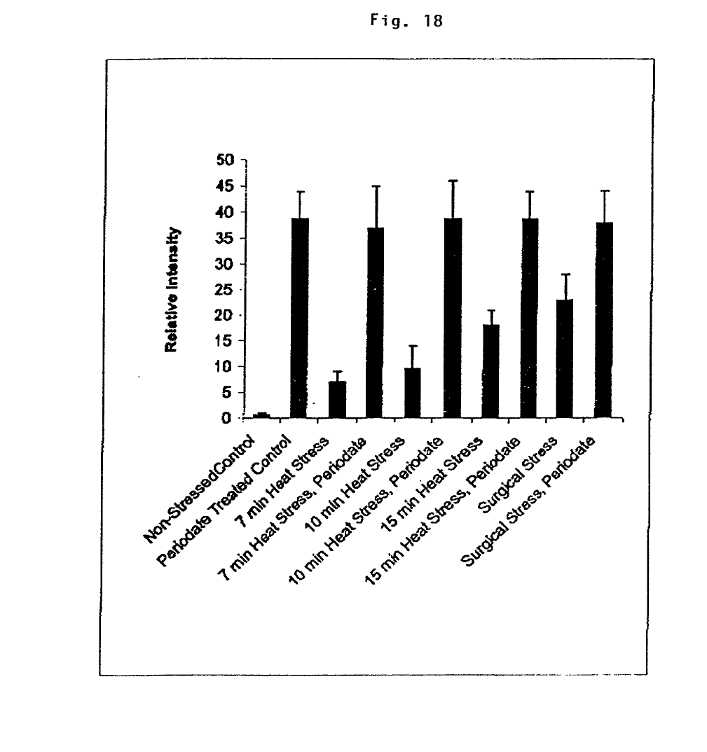

1. Different stress treatments activate the HIF in blood serum to different extent. However, when serum samples from differently stressed mice were treated with periodate, the Hsp70-inducing activity further increased in all samples to a level identical to that seen in periodate-activated serum from non-stressed mice, indicating that stress-inducing and oxidizing agent-generated Hsp-inducing activity is the same. This is shown by FIG. 18.

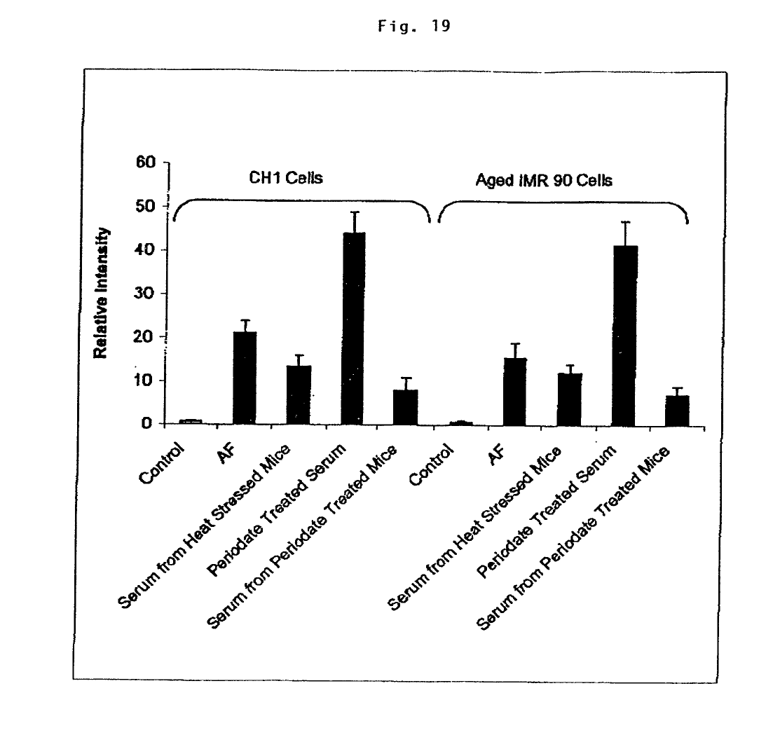

2. In all four cases (with AF, serum from stressed mice, periodate-treated serum from non-stressed mice, and serum from periodate-injected mice) the activity was capable of inducing a robust expression of Hsp70 in CH 1 cells and aged human fibroblasts (both incapable of a substantial induction of Hsp70 in response to stresses). This is shown by FIG. 19.

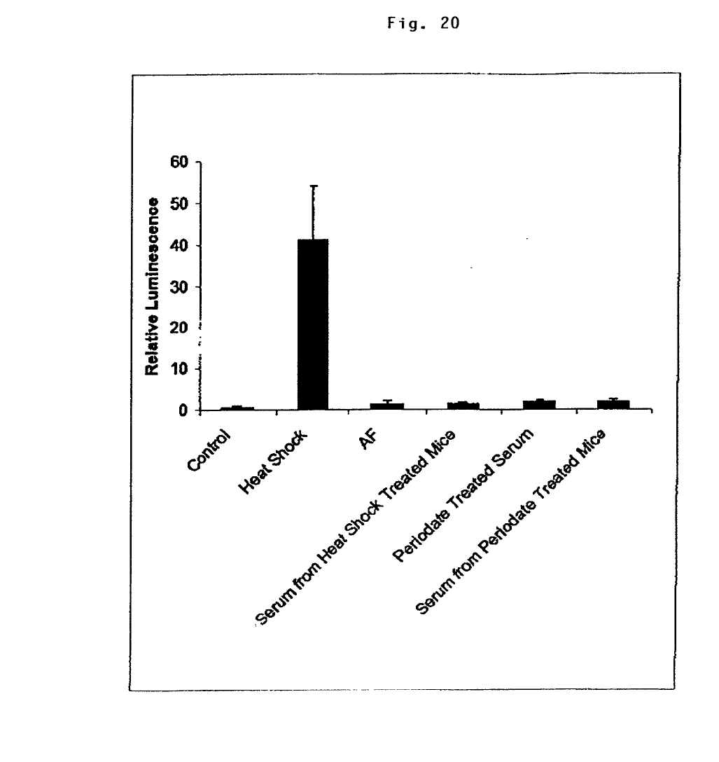

3. In all four cases (with AF, serum from stressed mice, periodate-treated serum from non-stressed mice, and serum from periodate-injected mice) the HIF activity was incapable of inducing in BRK cells the expression of recombinant luciferase controlled by a short (320nt) Hsp70 promoter that contains heat shock element (HSE) and is sufficient for activation of luciferase expression by heat shock and other stresses. This is shown by FIG. 20. Whereas heat shock of cells strongly induces the expression of luciferase under truncated Hsp70 promotor—very little, if any, expression is seen with HIF. The results with luciferase substantiates the notion that the induction of Hsp70 expression by the HIF occurs via a pathway different from a regular stress-response pathway and that the recognition element involved in the response to the HIF is probably located more than 320 nucleotides upstream from Hsp70 gene.

4. In all four cases (with AF, serum from stressed mice, periodate-treated serum from unstressed mice, and serum from periodate-injected mice), when fractionated on HPLC gel filtration column, the HSP inducing activity migrates as a protein of approximately 57 kDa.

The present invention is not to be limited in scope nor restricted in form except by the claims appended hereto.

What I claim is:

1. A method for in-situ generation of an active heat shock protein inducing factor capable of inducing expression of at least one heat shock protein species intracellularly, said method comprising the steps of:
obtaining access in-situ to a proteinaceous inactive form of heat shock protein-inducing factor, said proteinaceous inactive form being a naturally occurring composition in the blood serum of living mammals;
introducing an agent in sufficient quantity to said proteinaceous inactive form in-situ to initiate an activating reaction; and
permitting said activating reaction to proceed such that active heat shock protein-inducing factor is generated as a reaction product, said activated heat shock protein-inducing factor being able to induce cellular expression of at least one species of heat shock protein as a protection against injurious stress.

2. A method for in-situ generation of an active heat shock protein inducing factor capable of inducing expression of at least one heat shock protein species intracellularly within mammalian cells, said method comprising the steps of:
obtaining access in-situ to a proteinaceous inactive form of heat shock protein-inducing factor, said proteinaceous inactive form being a naturally occurring composition in the blood serum of living mammals;
introducing an agent in sufficient quantity to said proteinaceous inactive form in-situ to initiate an activating reaction;
permitting said activating reaction to proceed such that active heat shock protein-inducing factor is generated as a reaction product; and
interacting said generated, active heat shock protein-inducing factor with living mammalian cells, said interaction causing an induced expression of at least one species of heat shock protein by the mammalian cells as a protection against injurious stress.

3. The method for in-situ generation as recited in claim 1 or 2 wherein said agent is selected from the group consisting of oxidizing chemical compounds and their derivatives.

4. The method for in-situ generation as recited in claim 1 or 2 wherein said agent is selected from the group consisting of acids and acid derivatives.

5. The method for in-situ generation as recited in claim 1 or 2 wherein said agent is selected from the group consisting of deglycolsilating agents and their derivatives.

6. The method for in-situ generation as recited in claim 1 or 2 wherein said agent is selected from the group consisting of enzymes capable of modifying, removing or transferring glycosilic groups.

7. The method for in-situ generation as recited in claim 1 or 2 wherein said method is used in-vivo.

8. The method for in-situ generation as recited in claim 1 or 2 wherein said method is used in-vitro.
